(19) United States
(12) Patent Application Publication
(10) Pub. No.: US 2015/0284710 A1
(43) Pub. Date: Oct. 8, 2015

(54) METHOD OF MANIPULATING SOLID CARRIERS AND AN APPARATUS OF MANIPULATING SOLID CARRIERS

(71) Applicant: Seiko Epson Corporation, Tokyo (JP)

(72) Inventors: Toshiro Murayama, Suwa (JP); Yuji Saito, Suwa (JP); Fumio Takagi, Suwa (JP)

(73) Assignee: SEIKO EPSON CORPORATION, Tokyo (JP)

(21) Appl. No.: 14/441,327

(22) PCT Filed: Nov. 12, 2013
(86) PCT No.: PCT/JP2013/006657

§ 371 Date: May 7, 2015

(30) Foreign Application Priority Data
Nov. 12, 2012 (JP) 2012-248320

(51) Int. Cl. C12N 15/10; C12Q 1/68

(52) U.S. Cl. CPC C12N 15/1013; C12Q 1/6806

(57) ABSTRACT

The present invention provides a method of manipulating a solid carrier and an apparatus of manipulating a solid carrier in which the solid carrier is passed through more regions in liquid in a vessel. More specifically, the method of manipulating the solid carrier is a method of manipulating a solid carrier in a liquid contained in a vessel, the solid carrier being magnetic and capable of carrying a substance, the method comprising: manipulating the solid carrier using a magnetic force application unit adapted to applying a magnetic force to the solid carrier in the vessel so that the relative position between the magnetic force application unit and the vessel is altered over a period of time by means of directing the solid carrier in a direction along either one or a combination of the vectors of the longitudinal direction of the vessel and the direction crossing the longitudinal direction of the vessel.

### TECHNICAL FIELD

Cross reference to related applications: This application claims the benefit of Japanese Patent Application No. 2012-248320 filed on Nov. 12, 2012, the entire disclosure of which is hereby incorporated by reference.

The present invention relates to a method of manipulating solid carriers and an apparatus of manipulating solid carriers.

### BACKGROUND ART

Medical treatments using genes, such as genetic diagnosis or gene therapies, have received more attentions because of the advances in gene technologies in recent years. Besides, many approaches have been developed which use genes for breeding and breed identification in the agriculture and livestock fields. PCR (polymerase chain reaction) has widely been used as a technique for making use of the genes. In recent years, PCR is an essential tool for revealing information of biological substances. In PCR, by subjecting a solution (reaction solution) containing a nucleic acid to be amplified (target nucleic acid) and a reagent to thermal cycles, the target nucleic acid is amplified. Typical thermal cycles for PCR are performed at two or three different temperatures.

On the other hand, kits for a simple and rapid test such as immunochromatography have been the mainstream for the current diagnosis of an infectious disease including influenza in medical practices. Such simple and rapid tests, however, often suffer from poor accuracy and it has thus been desired to apply PCR, which can be expected to provide diagnosis with higher accuracy, to the diagnosis of the infectious diseases. Furthermore, only short period of time can be used for the medical check in general outpatient practices in a medical facility because of the limited time available for clinical examinations. Therefore, current examinations of, for example, influenza viruses are performed rapidly using an easy approach such as immunochromatography at the expense of the accuracy of the examinations.

Under such circumstances, in the field of medical practices, it is necessary to reduce the time required for reactions in order to achieve PCR examinations which can be expected to provide a result with higher accuracy. As a device for achieving PCR reactions in a short period of time, for example, some methods are disclosed as a technique of PCR, such as a method using magnetic beads (Japanese Patent Laid-open No. 2009-207459), a method of performing thermal cycles of PCR using magnetic beads as a tool for moving liquid droplets which are moved in a region on a substrate where the temperature is changed (Japanese Patent Laid-open No. 2008-012490), and a method of extracting nucleic acids, by pouring magnetic particles and reagents into a vessel having a shape of a funnel and externally forming a magnetic field using two or more magnets to manipulate magnetic particles in the vessel, and performing different reactions by replacing the reagents in the vessel with a pipette at the same time as manipulating the magnetic particles (Japanese Patent Publication No. 2008-507705T).

According to the Japanese Patent Laid-open No. 2009-207459, biological samples that travel in a flow passage along with a fluid are adsorbed onto magnetic particles. The magnetic particles are used for breaking up the biological samples. In this case, a magnetic force is applied to the magnetic particles from the outside of the flow passage to manipulate or hold the magnetic particles at a predetermined position. In addition, this publication also describes a method of adsorbing nucleic acids onto the magnetic particles held in the flow passage. In these methods, the magnetic particles are used as obstacles that can be manipulated. Biological substances are carried by a solution flowing through the flow passage. This causes problems of disposal of wasted liquid and difficult mixing of the reagents.

Japanese Patent Laid-open No. 2008-012490 describes a method of externally applying a magnetic force to liquid droplets containing magnetic particles encapsulated therein to move the liquid droplets across points on a substrate that are different in temperature, by using a physical force exerted between the magnetic particles and their surrounding liquid, to perform PCR. The publication also describes a method of adsorbing nucleic acids onto the magnetic particles and moving them between different reagents to adsorb and wash the nucleic acids in a similar way. In this publication, the magnetic particles are encapsulated in the liquid droplets having a free interface, and they are moved in a direction horizontal to the surface of the substrate. However, the amount of the liquid that can be present around the magnetic particles is limited and the motion of the magnetic particles and the liquid is completely synchronous with each other. It seems that the magnetic particles are washed with the reagents only in a limited region for washing, and it is thus hard to improve the efficiency of the washing operation.

Japanese Patent Publication No. 2008-507705T uses more than one magnets for the magnetic particles placed in a vessel to change, over a period of time, the position near the vessel receiving the largest magnetic force to manipulate the magnetic particles in the vessel. Efficiency in the steps of washing and elution required for the extraction of nucleic acids is thus improved. In addition, the magnetic particles in this publication are shaken in a predetermined region in the vessel. The surrounding reagent is replaced using a suction/injection device such as a pipette to achieve several washing operations. This method requires troublesome operations to replace the reagent, separate the magnetic particles and the reagent for the replacement, or hold them appropriately, which could have an adverse effect on improving the efficiency of the operation.

### SUMMARY OF INVENTION

### Technical Problem

The present invention was made in order to overcome at least some of the aforementioned problems and can be implemented as the following modes or embodiments.

[Embodiment 1] A method of manipulating a solid carrier according to this embodiment is a method of manipulating a solid carrier in a liquid contained in a vessel, the solid carrier being magnetic and capable of carrying a substance, the method comprising: manipulating the solid carrier using a magnetic force application unit adapted to applying a magnetic force to the solid carrier in the vessel so that the relative position between the magnetic force application unit and the vessel is altered over a period of time by means of directing the solid carrier in a direction along either one or a combination of the vectors of the longitudinal direction of the vessel and a cross-sectional direction crossing the longitudinal direction of the vessel.

According to this embodiment, the solid carrier in the vessel is manipulated by changing the relative position between the magnetic force application unit and the vessel over a period of time, and changing the magnetic field near the solid carrier and the vessel over a period of time. The solid carrier is moved in the vessel in the longitudinal and horizontal directions of the vessel to thereby allowing the solid carrier in the liquid in the vessel to pass through more regions.

[Embodiment 2 ] In the method of manipulating a solid carrier as described in the aforementioned embodiment, the magnetic force application unit comprises at least one permanent magnet.

According to this embodiment, heat is less likely to be generated, so that the solid carrier can be directed in an efficient manner.

[Embodiment 3 ] In the method of manipulating a solid carrier as described in the aforementioned embodiments, the position of the solid carrier is changed in the vessel by moving the permanent magnet to a position of the peripheral surface of the vessel and then moving the permanent magnet away from the position to move the permanent magnet closer to another position of the peripheral surface of the vessel.

According to this embodiment, the solid carrier can be directed in an efficient manner.

[Embodiment 4 ] In the method of manipulating a solid carrier as described in the aforementioned embodiments, the solid carrier is directed using two permanent magnets that are opposed to each other at a certain distance.

According to this embodiment, the solid carrier can be directed in a more efficient manner.

[Embodiment 5 ] In the method of manipulating a solid carrier as described in the aforementioned embodiments, the solid carrier is directed within the vessel as a result of the relative rotation between the magnetic force application unet and the vessel.

According to this embodiment, the solid carrier is moved along the inner surface of the vessel. By changing the distance between the permanent magnet and the vessel, the magnitude of the magnetic force near the vessel is altered. The partices of the solid carrier are repeatedly gathered and dispersed in the vessel. As a result, the solid carrier is floated in many regions in the liquid, which improves the efficiency of the washing operation.

[Embodiment 6 ] In the method of manipulating a solid carrier as described in the aforementioned embodiments, at least three permanent magnets are provided, and the solid carrier is directed in at least two directions in a plane perpendicular to the longitudinal direction of the vessel as a result of the relative displacement of the position between the vessel and the permanent magnets.

According to this embodiment, the rocking motion of a permanent magnet for use in externally applying a magnetic force is performed in two- and three-dimensional manners. The displacement can thus be performed efficiently.

[Embodiment 7 ] An apparatus of manipulating a solid carrier according to this embodiment is an apparatus of manipulating a solid carrier in a liquid contained in a vessel, the solid carrier being magnetic and capable of carrying a substance, the apparatus comprising: a magnetic force application unit adapted to applying a magnetic force to the solid carrier in the vessel, the solid carrier being manipulated so that the relative position between the magnetic force application unit and the vessel is altered over a period of time by means of directing the solid carrier in a direction along either one or a combination of the vectors of the longitudinal direction of the vessel and a cross-sectional direction crossing the longitudinal direction of the vessel.

According to this embodiment, the solid carrier is manipulated in the vessel by changing the relative position between the magnetic force application unit and the vessel, and changing the magnetic field near the solid carrier and the vessel over a period of time. In this way, it is possible to provide an apparatus of manipulating a solid carrier in which the relative position of the magnetic force application unit and the vessel is changed over a period of time, and the solid carrier is moved in the vessel in the longitudinal and horizontal directions of the vessel, thereby to allow the solid carrier to pass through more regions in the liquid in the vessel.

[Embodiment 8 ] A method of manipulating a solid carrier according to this embodiment is a method of manipulating a magnetic solid carrier in a vessel comprising liquid, the vessel having a longitudinal direction, the method comprising the step of: altering a relative position between the magnetic force application unit adapted to applying a magnetic force to the solid carrier from outside of the vessel and the vessel over a period of time, to direct the solid carrier, in one of a planar direction crossing the longitudinal direction of the vessel, the longitudinal direction of the vessel, and a direction determined by combining the vectors of the longitudinal direction of the vessel and the planar direction crossing the longitudinal direction of the vessel.

According to this embodiment, the solid carrier is manipulated in the vessel by changing the relative position between the magnetic force application unit and the vessel over a period of time, and changing the magnetic field near the solid carrier and the vessel over a period of time. The solid carrier can be passed through more regions in the liquid in the vessel by moving the solid carrier in the vessel in one of the planar direction crossing the longitudinal direction of the vessel, the longitudinal direction of the vessel, and the direction determined by combining the vectors of the longitudinal direction of the vessel and the planar direction crossing the longitudinal direction of the vessel.

[Embodiment 9 ] A method of manipulating a solid carrier according to this embodiment is the method of manipulating a solid carrier according to the Embodiment 8, wherein: the magnetic force application unit comprises at least one permanent magnet.

According to this embodiment, heat is less likely to be generated, so that the solid carrier can be directed in an efficient manner.

[Embodiment 10 ] A method of manipulating a solid carrier according to this embodiment is the method of manipulating a solid carrier according to the Embodiment 8 or 9, comprising: moving the magnet closer to a first position of the vessel; moving the magnet away from the first position; moving the magnet closer to a second position of the vessel, the second position being different from the first position; and moving the magnet away from the second position, wherein the solid carrier is thereby directed in the vessel.

According to this embodiment, the solid carrier can be directed in an efficient manner.

[Embodiment 11 ] A method of manipulating a solid carrier according to this embodiment is the method of manipulating a solid carrier according to any one of the Embodiments 8 to 10, wherein: the magnet is two permanent magnets that are opposed to each other at a certain distance.

According to this embodiment, the solid carrier can be directed in a more efficient manner.

[Embodiment 12 ] A method of manipulating a solid carrier according to this embodiment is the method of manipulating a solid carrier according to the Embodiment 11, comprising: decreasing a relative distance between a first magnet and the vessel while increasing a relative distance between a second magnet and the vessel, the second magnet being opposed to the first magnet; and increasing the relative distance between the first magnet and the vessel while decreasing the relative distance between the second magnet and the vessel, wherein the solid carrier is directed in the vessel thereby.

According to this embodiment, the solid carrier can be directed in a more efficient manner.

[Embodiment 13 ] A method of manipulating a solid carrier according to this embodiment is the method of manipulating a solid carrier according to the Embodiment 8 or 9, wherein the solid carrier is directed in the vessel by means of rotating the magnet around the vessel.

According to this embodiment, the solid carrier is moved along the inner surface of the vessel. By changing the distance between the permanent magnet and the vessel, the magnitude of the magnetic force near the vessel is altered. The particles of the solid carrier are repeatedly gathered and dispersed in the vessel. As a result, the solid carrier is floated in many regions in the liquid, which improves the efficiency of the washing operation.

[Embodiment 14 ] A method of manipulating a solid carrier according to this embodiment is the method of manipulating a solid carrier according to the Embodiment 8 or 9, wherein at least three permanent magnets are provided, and one of a first distance, second distance, and a third distance being determined to be smaller than the remaining two distances, the first distance representing a distance between the vessel and a first magnet, the second distance representing a distance between the vessel and a second magnet, and the third distance representing a distance between the vessel and a third magnet; and the solid carrier is thereby directed in a plane perpendicular to the longitudinal direction of the vessel.

According to this embodiment, the rocking motion of a permanent magnet for use in externally applying a magnetic force is performed in two- and three-dimensional manners. The displacement can thus be performed efficiently.

[Embodiment 15 ] An apparatus of manipulating a solid carrier according to this embodiment is an apparatus of manipulating a magnetic solid carrier in a vessel comprising liquid, the vessel having a longitudinal direction, the apparatus comprising: magnetic force application unit adapted to applying a magnetic force to the solid carrier from outside the vessel, wherein the magnetic force application unit is manipulated so that the relative position between the magnetic force application unit and the vessel is altered over a period of time, to direct the solid carrier, by using the magnetic force application unit, in one of a planar direction crossing the longitudinal direction of the vessel, the longitudinal direction of the vessel, and a direction determined by combining the vectors of the longitudinal direction of the vessel and the planar direction crossing the longitudinal direction of the vessel.

According to this embodiment, the solid carrier is manipulated in the vessel by changing the relative position between the magnetic force application unit and the vessel, and changing the magnetic field near the solid carrier and the vessel over a period of time. In this way, it is possible to provide an apparatus of manipulating a solid carrier in which the relative position of the magnetic force application unit and the vessel is changed over a period of time, and the solid carrier is moved in the vessel in the longitudinal and horizontal directions of the vessel, thereby to allow the solid carrier to pass through more regions in the liquid in the vessel.

[Embodiment 16 ] An apparatus of manipulating a solid carrier according to this embodiment is the apparatus of manipulating a solid carrier according to the Embodiment 15, wherein the vessel is a tube having a first oil plug, a washing solution plug, a second oil plug, an elution solution plug, and a third oil plug in the longitudinal direction thereof.

According to this embodiment, the nucleic acids can easily be purified from a sample.

### BRIEF DESCRIPTION OF DRAWINGS

FIG. 1 is a perspective view showing an example of an apparatus for the extraction of nucleic acids according to the Embodiment 1.

FIG. 2 is a view diagrammatically showing essential parts of a device for the extraction of nucleic acids according to the Embodiment 1.

FIG. 3 is a view diagrammatically showing essential parts of the device for the extraction of nucleic acids according to a modified version.

FIG. 4 is a view diagrammatically showing essential parts of the device for the extraction of nucleic acids according to a modified version.

FIG. 5 is a view diagrammatically showing a device for the extraction of nucleic acids according to a modified version.

FIG. 6 a view diagrammatically showing essential parts of the device for the extraction of nucleic acids according to a modified version.

FIG. 7 is a view diagrammatically showing an example of a kit for the extraction of nucleic acids according to the Embodiment 1.

FIG. 8 is a view diagrammatically showing an example of a kit for the extraction of nucleic acids according to the Embodiment 1.

FIG. 9 is a view showing a method of externally applying a magnetic force to move magnetic particles according to the Embodiment 1.

FIG. 10 is a view showing a method of externally applying a magnetic force to move magnetic particles according to the Embodiment 1.

FIG. 11 is a view showing a method of externally applying a magnetic force to move magnetic particles according to the Embodiment 1.

FIG. 12 is a graph showing the results obtained in the Experiment 1.

FIG. 13 is a graph showing the relationship between the elusion temperature and the yield of DNAs according to the Experiment 4.

FIG. 14 is a view showing an exemplified configuration of a permanent magnet and a tube according to the Embodiment 2.

FIG. 15 is a view showing an exemplified configuration of the permanent magnet and the tube according to the Embodiment 2.

FIG. 16 is a view showing an exemplified configuration of permanent magnets and a tube according to the Embodiment 3.

FIG. 17 is a perspective view showing an example of an apparatus for the extraction of nucleic acids according to the modified version 3.

FIG. 18 is a view diagrammatically showing a device for the extraction of nucleic acids according to a modified version 4.

FIG. 19 is a diagrammatic representation for use in illustrating a method for the extraction of nucleic acids according to a modified version 5.

### DESCRIPTION OF EMBODIMENTS

Now, some embodiments of the present invention are described. The embodiments described below are for illustrating examples of the present invention. The present invention is not limited to the following embodiments, and includes various modifications that can be achieved without changing the scope of the present invention. The components and parts described below are not necessarily essential ones for the present invention.

An apparatus for the extraction of nucleic acids is described as an embodiment of an apparatus of manipulating a solid carrier for use in extracting nucleic acids with a kit for the extraction of nucleic acids being mounted thereon.

### Embodiment 1

1. Apparatus for the Extraction of Nucleic Acids

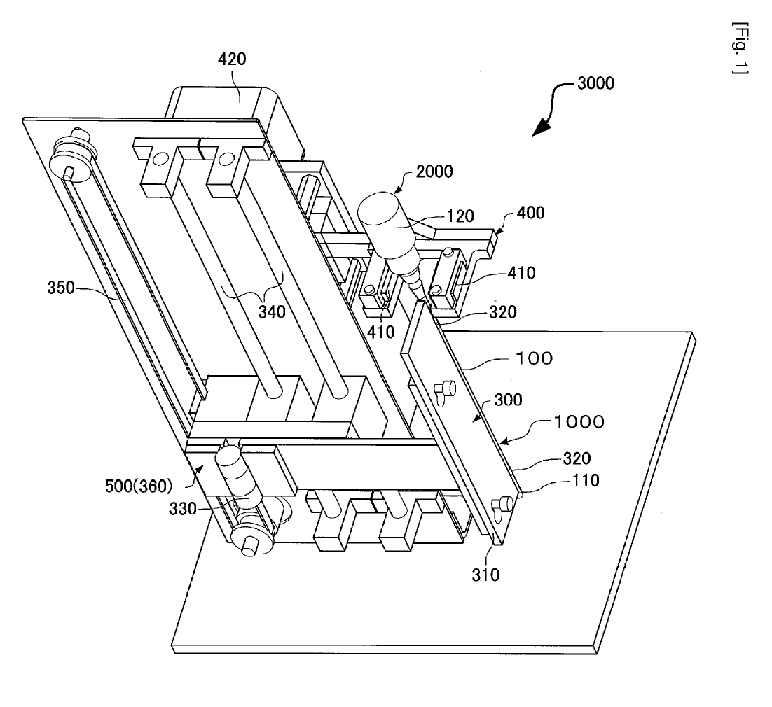

FIG. 1 is a perspective view showing an example of an apparatus for the extraction of nucleic acids according to the present embodiment. An apparatus for the extraction of nucleic acids 3000 of this embodiment can suitably be applied to a kit for the extraction of nucleic acids, a device for the extraction of nucleic acids, and a method for the extraction of nucleic acids, which are described later.

The apparatus for the extraction of nucleic acids 3000 of this embodiment comprises a tube mount 300, a magnetic force application unit 400, and a moving mechanism 500. The tube mount 300 receives a tube (vessel) 100 that forms a device for the extraction of nucleic acids 1000. The magnetic force application unit 400 externally applies a magnetic force to the tube 100, in particular, from the side of the tube 100 after the tube 100 is mounted on the tube mount 300. The moving mechanism 500 changes the relative position of the tube mount 300 and the magnetic force application unit 400 in the longitudinal direction of the tube 100.

The tube 100 mounted on the tube mount 300 of the apparatus for the extraction of nucleic acids 3000 is described later.

The tube mount 300 is a place where the tube 100 is mounted. An adsorption chamber 120 connected to the tube 100 may also be mounted on the tube mount 300 along with the tube 100. A structure and a mechanism for receiving the tube 100 in the tube mount 300 may appropriately be designed as long as a magnetic force can be applied to the tube 100 and, if necessary, to the adsorption chamber 120 by the magnetic force application unit 400. The tube mount 300 may be configured so that the tube 100 is stretched straight and supported by the tube mount 300 when the tube 100 is flexible and has a curved or bent posture. The tube mount 300 in the illustrated embodiment has a support plate 310. The tube 100 is disposed against the support plate 310. Although the support plate 310 is not an essential element, it can suppress the vibration of the tube 100. In addition, the tube mount 300 in the illustrated embodiment has clip mechanisms 320 for securing the tube 100 at two positions.

The tube mount 300 and the magnetic force application unit 400 are configured so that their relative position is changed in the longitudinal direction of the tube 100. Therefore, when the tube mount 300 is designed so as to move relative to the magnetic force application unit 400 without any movement of the latter, the apparatus for the extraction of nucleic acids 3000 has a moving mechanism 360 as the moving mechanism 500 to move the tube mount 300, as illustrated. Alternatively, when the magnetic force application unit 400 has a moving mechanism, the moving mechanism 360 of the tube mount 300 may be omitted. In the illustrated embodiment, the tube mount 300 comprises a hinge 330, a guiding rail 340, a drive belt 350, and a motor which is not shown.

Although the apparatus for the extraction of nucleic acids 3000 in this embodiment has one tube mount 300, it may have two or more tube mounts. In such a case, two or more magnetic force application units 400 may be provided. The two or more tube mounts 300 may be configured so that they function independently or in cooperation with each other.

The magnetic force application unit 400 is configured to apply a magnetic force to the tube 100 and, if necessary, to the adsorption chamber 120 after the tube 100 is mounted on the tube mount 300. The magnetic force application unit 400 comprises, for example, a permanent magnet, an electromagnet, or a combination thereof. Although the magnetic force application unit 400 has at least one magnet, two or more magnets may be provided. It is preferable that a permanent magnet is used for the magnetic force application unit because this configuration is less likely to produce heat in comparison to a case where an electromagnet is used. The permanent magnet used may be, for example, a nickel-based, iron-based, cobalt-based, samarium-based, or neodymium-based permanent magnet.

The magnetic force application unit 400 can apply a magnetic force to magnetic particles (solid carriers) M provided in the adsorption chamber 120 and the tube 100. By changing the relative position between the tube mount 300 and the magnetic force application unit 400, the magnetic particles M can be moved in the adsorption chamber 120 and the tube 100.

In the illustrated embodiment, the magnetic force application unit 400 has a pair of permanent magnets 410. The permanent magnets 410 are opposed to each other relative to the adsorption chamber 120 and the tube 100. The permanent magnets 410 are separated from each other at a distance that is larger than the diameter of the tube 100. The direction of the polarity of the permanent magnets 410 is not specifically limited. The magnetic force application unit 400 is configured so that the relative position of the magnetic force application unit 400 and the tube mount 300 is changed in the longitudinal direction of the tube 100. When the magnetic force application unit 400 is designed to be moved relative to the tube mount 300 without any movement of the latter, the apparatus for the extraction of nucleic acids 3000 has a moving mechanism as the moving mechanism 500 to move the magnetic force application unit 400.

In the illustrated embodiment, the magnetic force application unit 400 is configured so that, when one of the permanent magnets 410 approaches the tube 100, the other leaves from the tube 100. A motor 420 is used to move the pair of permanent magnets 410 laterally relative to the tube 100. Operation of the motor 420 causes the magnetic particles M to rock laterally in the tube 100 in the direction crossing the longitudinal direction of the tube 100.

The motor 420 can be operated at any appropriate time regardless of which part of the adsorption chamber 120 or the tube 100 a magnetic force is applied to. It is, however, noted that the efficiencies of washing and eluting the magnetic particles M in the tube 100 can be increased by operating the motor 420 when the permanent magnets 410 are positioned at the level of the second plug 20 or the fourth plug 40 in the tube 100.

Each magnetic particle M has magnetism and can carry a substance. The magnetic particles M are manipulated in a solution contained in the tube 100. More specifically, the magnetic particles M are manipulated so that the relative position of the magnetic force application unit 400 and the magnetic particles M is altered over a period of time by directing the magnetic particles M in the tube 100 in one of (1) the longitudinal direction of the tube 100, (2) a cross-sectional direction crossing the longitudinal direction of the tube 100, and (3) a direction determined by combining the vectors of the longitudinal direction of the tube 100 and the cross-sectional direction crossing the longitudinal direction of the tube 100, or in one of (1) a lateral direction crossing the longitudinal direction of the tube 100, (2) the longitudinal direction of the tube 100, and (3) a direction determined by combining the vectors of the longitudinal direction of the tube 100 and the lateral direction crossing the longitudinal direction of the tube 100, by using the magnetic force application unit 400 for applying a magnetic force to the magnetic particles M.

What is required for manipulating the magnetic particles M in the tube 100 is to change the relative position of the magnetic force application unit 400 and the tube 100. This can be achieved by keeping either of the tube 100 or the magnetic force application unit 400 stationary and moving the other, or alternatively, by moving both of the tube 100 and the magnetic force application unit 400. This embodiment describes a case where the tube 100 is fixed after being mounted on the tube mount 300 and the magnetic force application unit 400 is moved.

In this embodiment, a “plug” of a liquid refers to a portion of an inner space of the vessel (tube 100) that is substantially filled with the subject liquid alone. The inner space of the vessel is partitioned longitudinally by the plugs. As used herein, the term “substantially” indicates that a few amount (e.g., as a thin film or foam) of another substance (e.g., a liquid or a gas) may be present around the plug, that is, on the inner wall of the vessel or on the interface of the plug. In addition, the vessel refers to a hollow component that may be deformed. The vessel has a section with such a size that allows the liquid to keep the shape as the plug within the capillary.

The apparatus for the extraction of nucleic acids 3000 of this embodiment allows the automation of a pre-treatment of PCR, which significantly reduces the time and labor required for the pre-treatment. In addition, the magnetic force application unit 400 can be rockd in the apparatus for the extraction of nucleic acids 3000 of this embodiment. This provides more efficient washing (purification) of the magnetic particles M carrying the nucleic acids adsorbed thereon, further improving the accuracy of PCR.

2. Device for the Extraction of Nucleic Acids

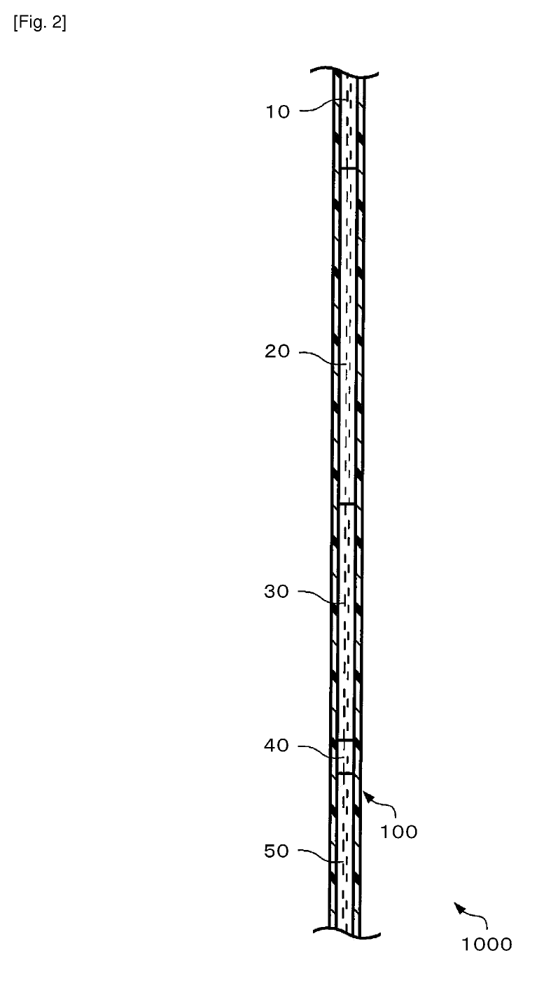

FIG. 2 is a view diagrammatically showing essential parts of the device for the extraction of nucleic acids 1000 according to this embodiment.

The device for the extraction of nucleic acids 1000 has the tube 100, a first plug 10, a second plug 20, a third plug 30, a fourth plug 40, and a fifth plug 50. The device for the extraction of nucleic acids 1000 has the longitudinal direction and has the tube (vessel) 100 in which the first plug 10 made of an oil (liquid), the second plug 20 made of a first washing solution (liquid) that is phase separated from the oil, the third plug 30 made of an oil, the fourth plug 40 made of an elution solution (liquid) that is phase separated from the oil, and the fifth plug 50 is made of an oil are provided in this order. The tube 100 in this embodiment has the first through fifth plugs 10 to 50, but a sixth plug and a seventh plug, which are described later, may also be provided.

2.1. Tube

The tube 100 forms an essential part of the device for the extraction of nucleic acids 1000. The device for the extraction of nucleic acids 1000 may comprise various components and parts other than the tube 100. The device for the extraction of nucleic acids 1000 may comprise, for example, a tubing connected to the tube 100, an stopper, a joint, a pump, or a controller, which are not shown herein.

The tube 100 is a hollow component having a cavity therein through which a liquid can be passed in its longitudinal direction. Although the tube 100 is straight in its longitudinal direction, it may be curved or bent. The shape and size of the cavity in the tube 100 are not specifically limited as long as the liquid can keep the shape of the plug in the tube 100. The size of the cavity in the tube 100 and the cross-sectional shape of the cavity perpendicular to the longitudinal direction of the tube 100 may be varied along the longitudinal direction of the tube 100. Whether the liquid can keep the shape of the plug in the tube 100 depends on factors including the material of the tube 100 and the type of the liquid. The cross-sectional shape of the tube 100 perpendicular to its longitudinal direction is appropriately designed to the extent that the liquid can keep the shape of the plug in the tube 100.

The shape of the outer periphery of the tube 100 as viewed in the cross section perpendicular to the longitudinal direction of the tube 100 is not specifically limited. The thickness (the length from the side of the inner cavity to the outer surface) of the tube 100 is not limited as well. When the cavity in the tube 100 has a circular cross section in the direction perpendicular to the longitudinal direction of the cavity, the inner diameter of the tube 100 (diameter of a circle in the cross section of the cavity perpendicular to the longitudinal direction thereof) may be, for example, equal to or larger than 0.5 mm but not larger than 3 mm. The tube 100 having an inner diameter within this range is preferable because a plug of liquid can be formed easily for a wide variety of materials of the tube 100 and for various liquids.

The material of the tube 100 is not specifically limited. Examples include glass, polymers, or metals. It is, however, preferable that a material such as glass or a polymer transparent to visible light is used for the tube 100 because the inside (within the cavity) can be observed from the outside of the tube 100. It is also preferable that a material that transmits the magnetic force or a non-magnetic material is used for the tube 100 because the application of the magnetic force from the outside of the tube 100 facilitates the migration of the magnetic particles M through the tube 100.

The first plug 10 made of a first oil, the second plug 20 made of the first washing solution that is phase separated from the oil, the third plug 30 made of a second oil that is phase separated from the first washing solution, the fourth plug 40 made of the elution solution that is phase separated from the oil, and the fifth plug 50 made of a third oil that is phase separated from the elution solution are provided in this order in the tube 100.

2.2. First Plug, Third Plug, and Fifth Plug

Each of the first plug 10, the third plug 30, and the fifth plug 50 is made of an oil. The oil of the first plug 10, the third plug 30, and the fifth plug 50 may be the same or different from each other. The oil may be selected from, for example, silicone oil such as dimethyl silicone, paraffin oil, mineral oil and a mixture thereof. The liquid of the first, second, third, fourth, and fifth plugs 10, 20, 30, 40, and 50 is selected so that the adjacent plugs are phase separated from each other.

The second plug 20 is provided between the first plug 10 and the third plug 30. A plug of another liquid may be provided adjacent to the first plug 10 opposite to the second plug 20. Although the first plug 10 preferably contains neither air bubbles nor any other liquid, air bubbles and another liquid may be present as long as the magnetic particles M carrying the nucleic acids adsorbed thereon can pass through the first plug 10. In addition, it is preferable that no air or liquid is present between the first plug 10 and the second plug 20. The air or another liquid may, however, be present as long as the magnetic particles M carrying the nucleic acids adsorbed thereon can pass from the first plug 10 to the second plug 20. Likewise, it is preferable that no air or liquid is present between the second plug 20 and the third plug 30. The air or another liquid may, however, be present as long as the magnetic particles M carrying the nucleic acids adsorbed thereon can pass from the second plug 20 to the third plug 30.

The fourth plug 40 is provided between the third plug 30 and the fifth plug 50. A plug of another liquid may be provided adjacent to the fifth plug 50 opposite to the fourth plug 40. Although the third plug 30 preferably contains neither air bubbles nor any other liquid, air bubbles and another liquid may be present as long as the magnetic particles M carrying the nucleic acids adsorbed thereon can pass through the third plug 30. In addition, it is preferable that no air or liquid is present between the third plug 30 and the fourth plug 40. The air or another liquid may, however, be present as long as the magnetic particles M carrying the nucleic acids adsorbed thereon can pass from the third plug 30 to the fourth plug 40. Likewise, it is preferable that no air or liquid is present between the fourth plug 40 and the fifth plug 50. The air or another liquid may, however, be present as long as the magnetic particles M carrying the nucleic acids adsorbed thereon can pass from the fourth plug 40 to the fifth plug 50. Furthermore, it is preferable that no air or liquid is present in the fifth plug 50.

The length (height) of the liquid column in the longitudinal direction of the tube 100 is not specifically limited as long as the first plug 10, the third plug 30, and the fifth plug 50 can be formed. The specific length of each of the first plug 10, the third plug 30, and the fifth plug 50 in the longitudinal direction of the tube 100 is equal to or longer than 1 mm but not longer than 50 mm. The length is preferably equal to or longer than 1 mm but not longer than 30 mm, and more preferably, equal to or longer than 5 mm but not longer than 20 mm, in order to avoid that a travel distance of the magnetic particles M becomes too long. Of these, when the length of the third plug 30 in the longitudinal direction of the tube 100 is long, the second plug 20 is less likely to be discharged in an aspect where the fourth plug 40 is discharged from the tube 100 on the side of the fifth plug 50. In this case, a specific length of the third plug 30 may be equal to or longer than 10 mm but not longer than 50 mm.

The first plug 10 and the fifth plug 50 each has a function of preventing the first washing solution forming the second plug 20 and the elution solution forming the fourth plug 40 from being contact with an external material (e.g., evaporation) or from being contaminated even when at least one end of the tube 100 is opened. The volume of the first washing solution and the elution solution can thus be kept constant even when at least one end of the tube 100 is exposed to the outside air. This contributes to reducing any fluctuation of the concentration of each liquid as well as contamination of each liquid. Accordingly, the nucleic acids and various other substances can be extracted more precisely during the extraction of the nucleic acids.

The third plug 30 serves to prevent the first washing solution forming the second plug 20 and the elution solution forming the fourth plug 40 from being mixed with each other. The third plug 30 may be made of an oil of a higher viscosity, which increases a “repellent effect” of the oil when the magnetic particles M are moved across the interface between the oil and the first washing solution forming the second plug 20. As a result, fewer water-soluble components on the surface of the magnetic particles M are brought into the oil forming the third plug 30 when the particles are moved from the plug of the first washing solution forming the second plug 20 to the oil forming the third plug 30.

2.3. Second Plug

The second plug 20 is positioned between the first plug 10 and the third plug 30 in the tube 100. The second plug 20 is made of the first washing solution. The first washing solution is a liquid that is phase separated from both the oil forming the first plug 10 and the oil forming the third plug 30. Examples of the first washing solution include water and a buffer having a solute at a concentration of 10 mM or lower, preferably 7 mM or lower, and more preferably, 5 mM or lower. The composition of the buffer is not specifically limited, and a tris-HCl buffer is an example. The buffer may contain EDTA (ethylenediaminetetraacetic acid). Such first washing solution allows efficient washing of the magnetic particles M carrying the nucleic acids adsorbed thereon.

The volume of the second plug 20 is not specifically limited and can appropriately be determined based on, for example, the amount of the magnetic particles M carrying the nucleic acids adsorbed thereon. For example, when the volume of the magnetic particles M is equal to 0.5 uL, the volume of 10 uL or larger is enough for the second plug 20. It is preferable that the volume is equal to or larger than 20 uL but not larger than 50 uL, and more preferably, equal to or larger than 20 uL but not larger than 30 uL. With the volume of the second plug 20 within this range, the magnetic particles M can be washed sufficiently when the volume of the magnetic particles M is equal to 0.5 uL. Although a larger volume of the second plug 20 is preferable for washing the magnetic particles M, the volume can appropriately be determined in consideration of the length and the width of the tube 100 as well as the length of the second plug 20 depending thereon in the longitudinal direction of the tube 100.

The second plug 20 may be made up of two or more smaller plugs separated from each other by a plug of an oil. When the second plug 20 is made up of two or more plugs separated from the oil plug(s), this means that two or more plugs of the first washing solution are formed. With the second plug 20 separated by the oil plug(s), the concentration of a water-soluble substance (in a case where such water-soluble substance is subjected to washing) achieved in the plugs of the first washing solution is smaller than the concentration of the water-soluble substance achieved in the single plug of the first washing solution of the same volume. The second plug 20 may be separated into any number of smaller plugs. For example, when the second plug 20 is divided into two smaller plugs having an equal volume, the concentration of the water-soluble substance (in a case where such water-soluble substance is subjected to washing) can be reduced to ¼ of the concentration obtained without separating the second plug 20 by calculation. The number of the divisions of the second plug 20 is determined appropriately in consideration of, for example, the length of the tube 100 and the substance to be washed.

2.4. Fourth Plug

The fourth plug 40 is positioned between the third plug 30 and the fifth plug 50 in the tube 100. The fourth plug 40 is made of the elution solution.

The elution solution is a liquid for use in releasing the nucleic acids adsorbed on the magnetic particles M from the magnetic particles M into the solution. Examples of the elution solution include purified water such as sterilized water, distilled water, or ion-exchanged water, or a solution of such water containing at least one enzyme, dNTP, probe, primer, or buffer. The elution solution is a liquid that is phase separated from both the oil forming the third plug 30 and the oil forming the fifth plug 50.

When water or an aqueous solution is used as the elution solution, the nucleic acids adsorbed on the magnetic particles M can be released (eluted) into the elution solution while the magnetic particles M carrying the nucleic acids adsorbed thereon are captured in the elution solution. When a solution in which at least one enzyme, dNTP, probe, primer or buffer is dissolved is selected as the elution solution, the nucleic acids adsorbed on the magnetic particles M can be released (eluted) and some or all of the components required for a reaction solution for PCR can be contained in the elution solution. This further reduces the time and labor in preparing a reaction solution for PCR using the elution solution. The concentration of at least one enzyme, dNTP, probe, primer or buffer dissolved in the elution solution forming the fourth plug 40 is not specifically limited and can be determined according to the reaction solution for PCR to be prepared.

It is noted that dNTP as used herein is a mixture of four kinds of deoxynucleotide triphosphates (dATP (deoxyadenosine triphosphate), dCTP (deoxycytidine triphosphate), dGTP (deoxyguanosine triphosphate), and dTTP (thymidine triphosphate).

The volume of the fourth plug 40 is not specifically limited and can appropriately be determined based on, for example, the amount of the magnetic particles M carrying the nucleic acids adsorbed thereon. For example, when the volume of the magnetic particles M is equal to 0.5 uL, the volume of 0.5 uL or smaller is enough for the fourth plug 40. It is preferable that the volume is equal to or larger than 0.8 uL but not larger than 5 uL, and more preferably equal to or larger than 1 uL but not larger than 3 uL. With the volume of the fourth plug 40 within this range, the nucleic acids can be eluted sufficiently from the magnetic particles M the when the volume of the magnetic particles M is equal to 0.5 uL. The volume of the fourth plug 40 for the elution of the nucleic acids from the magnetic particles M can appropriately be determined in consideration of the length and the width of the tube 100 as well as a rapid progress of a thermal cycle of PCR so that a heat capacity of the reaction solution does not increase excessively.

2.5. Effects

The device for the extraction of nucleic acids 1000 according to this embodiment has the tube 100 containing the oil, the first washing solution, and the elution solution provided as the plugs. Nucleic acids can thus be extracted in a significantly short period of time by introducing the magnetic particles M carrying the nucleic acids adsorbed thereon into the tube 100 through the first plug 10 and moving them to the fourth plug 40. More specifically, the magnetic particles M carrying the nucleic acids adsorbed thereon are introduced into the tube 100 through the first plug 10. They are passed through the oil forming the first plug 10, washed with the first washing solution forming the second plug 20, and then passed through the oil forming the third plug 30. The nucleic acids are released from the magnetic particles M in the elution solution forming the fourth plug 40. This means that the device for the extraction of nucleic acids 1000 of this embodiment can be used to obtain the elution solution containing the nucleic acids in a highly pure form by moving the magnetic particles M carrying the nucleic acids adsorbed thereon through the tube 100. Accordingly, the device for the extraction of nucleic acids 1000 can significantly reduce the time and labor required for the pre-treatment for PCR.

Modified Version

Configuration of the Device for the Extraction of Nucleic Acids

The device for the extraction of nucleic acids 1000 of this embodiment includes the tube 100, the first plug 10, the second plug 20, the third plug 30, the fourth plug 40, and the fifth plug 50. The device may, however, have a configuration with the addition of other function(s). The device for the extraction of nucleic acids of this embodiment may include a combination of the structures described below or a modified version of these structures.

End of the Tube

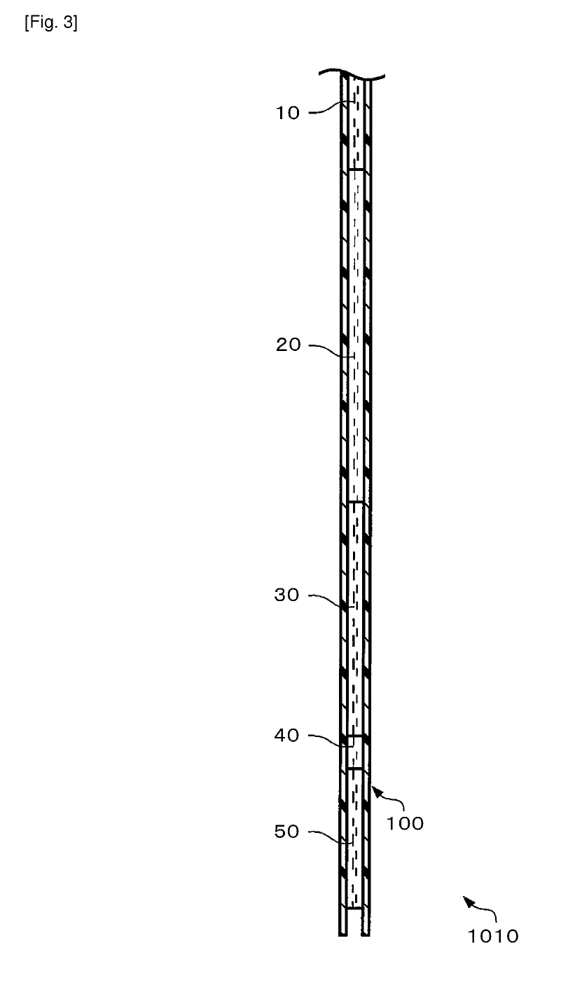

FIG. 3 is a view diagrammatically showing essential parts of the device for the extraction of nucleic acids according to this modified version. A device for the extraction of nucleic acids 1010 of this embodiment may have the tube 100 that is opened at the end at the side of the fifth plug 50. More specifically, as shown in FIG. 3, the tube 100 of the device for the extraction of nucleic acids 1010 is opened at the end at the side of the fifth plug 50. In this device for the extraction of nucleic acids 1010, the fifth plug 50 and the fourth plug 40 are discharged successively by applying a pressure to the inside of the tube 100 on the side of the first plug 10 in the tube 100. This allows for easy removal of the elution solution forming the fourth plug 40 containing the target nucleic acids into, for example, a reaction chamber for PCR using the device for the extraction of nucleic acids 1010.

Stopper

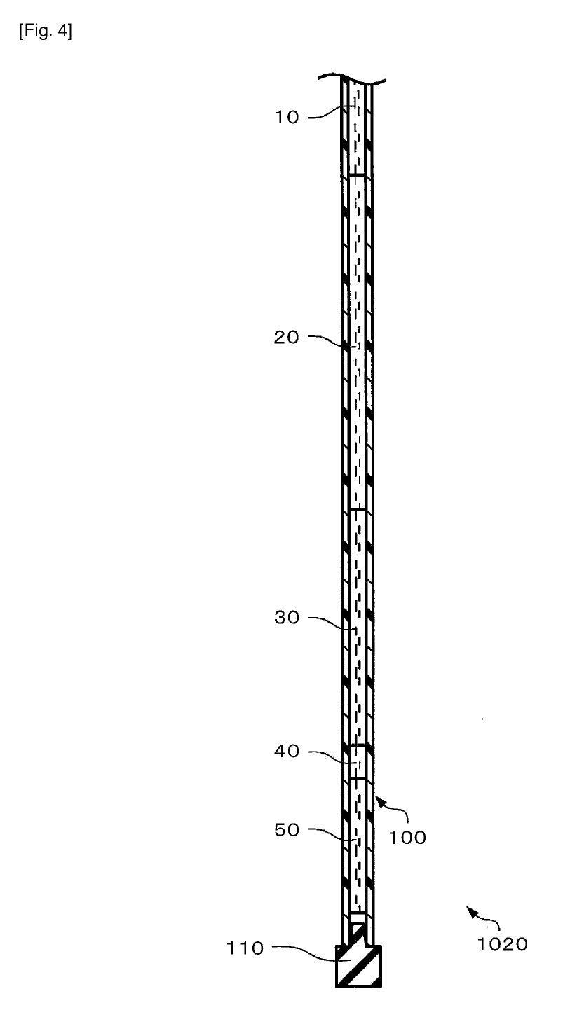

FIG. 4 is a view diagrammatically showing essential parts of the device for the extraction of nucleic acids according to this modified version. A device for the extraction of nucleic acids 1020 of this embodiment may further include, for example, a stopper 110 that can seal the end of the tube 100 at the side of the fifth plug 50 as shown in the figure. The stopper 110 can freely be fitted to and removed from the end of the tube 100. The stopper 110 may be made of, for example, a rubber, an elastomer, or a polymer. When the tube 100 is sealed with the stopper 110, the stopper 110 may contact with the fifth plug 50 or, alternatively, a gas such as air may be present between the fifth plug 50 and the stopper 110. In addition, while the stopper 110 can freely be fitted to and removed from the tube 100, a mechanism to achieve this is not specifically limited. In the embodiment shown in FIG. 4, a part of the stopper 110 is inserted into the tube 100 and secured thereto. The stopper 110 may, however, have a shape of a cap.

When the stopper 110 is removed from the device for the extraction of nucleic acids 1020, the end of the tube 100 at the side of the fifth plug 50 is opened. This corresponds to the device for the extraction of nucleic acids 1010 shown in FIG. 3. The device for the extraction of nucleic acids 1020 can thus be used for easy removal of the elution solution forming the fourth plug 40 containing the target nucleic acids into, for example, a reaction chamber for PCR. On the other hand, with the end of the tube 100 at the side of the fifth plug 50 sealed with the stopper 110 (as shown in FIG. 4), an effect of preventing each plug from being displaced in the tube 100 is provided. Accordingly, the plugs do not follow the movement of the magnetic particles M when the magnetic particles M are moved in the tube 100.

It is noted that the end of the tube 100 (at the side of the fifth plug 50) is sealed with the stopper 110 or connected to another vessel after a reagent is filled in the tube 100 in order to avoid any displacement of the fluid due to the gravity within the tube 100.

Adsorption Chamber

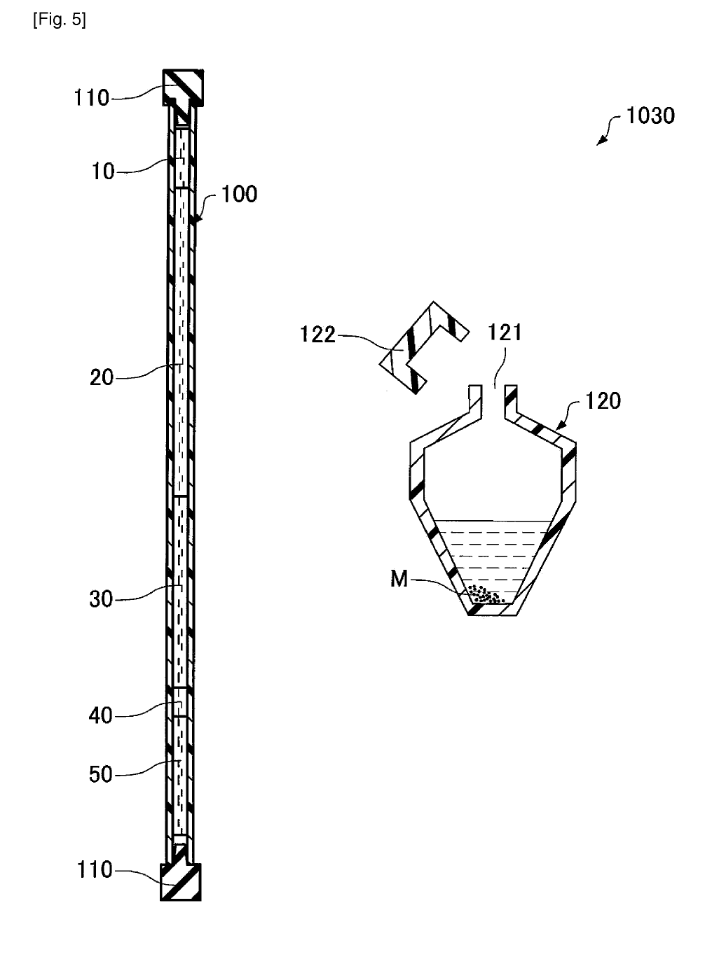

FIG. 5 is a view diagrammatically showing a device for the extraction of nucleic acids according to this modified version. As shown in FIG. 5, a device for the extraction of nucleic acids 1030 further includes an adsorption chamber (vessel) 120 that can freely be connected to and removed from the end of the tube 100 at the side of the first plug 10 in communication with each other.

The adsorption chamber 120 may be an independent member. The adsorption chamber 120 can contain a liquid. The adsorption chamber 120 has an opening 121 through which a liquid or a solid can be put into and removed from the adsorption chamber 120. In the embodiment shown in FIG. 5, the opening 121 in the adsorption chamber 120 is communicated with the end of the tube 100 at the side of the first plug 10. In addition, the adsorption chamber 120 may have two or more openings 121. One of such openings 121 may be communicated with the end of the tube 100 at the side of the first plug 10.

The inner volume of the adsorption chamber 120 is not specifically limited and, may be, equal to or larger than 0.1 mL but not larger than 100 mL. The opening(s) 121 of the adsorption chamber 120 may have a structure that can be sealed with a lid 122 when appropriate. The material of the adsorption chamber 120 is not specifically limited and may be, for example, a metal or a polymer.

The opening 121 in the adsorption chamber 120 can be connected to the end of the tube 100 at the side of the first plug 10. The type of the connection between the adsorption chamber 120 and the tube 100 is not specifically limited as long as the content therein does not escape therefrom. With the adsorption chamber 120 and the tube 100 connected, the inner space in the adsorption chamber 120 is communicated with the inner space in the tube 100. The adsorption chamber 120 may be removed from the tube 100, if necessary.

By providing the adsorption chamber 120 as in the case of the device for the extraction of nucleic acids 1030, the magnetic particles M, an adsorption solution, and a sample can be contained in the adsorption chamber 120 to allow the magnetic particles M to adsorb the nucleic acids. Then, the adsorption chamber 120 is connected to the end of the tube 100 at the side of the first plug 10. In this way, the magnetic particles M can easily be introduced into the tube 100 through the first plug 10.

The adsorption solution refers to a liquid in which nucleic acids are adsorbed onto the magnetic particles M. The adsorption solution is an aqueous solution containing, for example, a chaotropic agent. The adsorption solution may contain a chelating agent or a surfactant. More specifically, the adsorption solution may have disodium dihydrogen ethylenediamine tetraacetate or a dihydrate thereof dissolved therein or may contain, for example, polyoxyethylene sorbitan monolaurate.

The chaotropic agent as used herein refers to a substance that disrupts the intermolecular forces between water molecules and weakens the structure of the water molecules. Specific examples of the chaotropic agent include guanidinium ions, ureas, and iodide ions. When exposed to water containing the chaotropic agent, it is thermodynamically more favorable for the nucleic acids to be adsorbed onto a solid than being surrounded by water molecules. The nucleic acids in the water are thus adsorbed on the surface of the magnetic particles M. Examples of the substance having chaotropic properties in the water include guanidine hydrochloride and sodium iodide.

The adsorption chamber 120 can be shaken without being connected to the tube 100 to vigorously mix the liquid in the adsorption chamber 120. This allows quick adsorption of the nucleic acids onto the magnetic particles M. The adsorption chamber 120 may have the lid 122 that seals the opening 121. Furthermore, it is also possible to quantitatively concentrate the nucleic acids in the sample in the elution solution forming the fourth plug 40 by appropriately altering the amount of the sample to be introduced into the adsorption chamber 120 and the volume of the liquid (in particular, the fourth plug 40) in the tube 100.

By using an elastic material such as a rubber, an elastomer, or a polymer as a material of the adsorption chamber 120, the adsorption chamber 120 can be deformed and the inside of the tube 100 can be pressurized with the adsorption chamber 120 connected to the tube 100. In this way, a pressure can easily be applied to the tube 100 from the side of the first plug 10 in order to discharge the elution solution forming the fourth plug 40 from the end of the tube 100 at the side of the fifth plug 50. This configuration makes it possible to remove the elution solution into, for example, a reaction chamber for PCR.

The nucleic acids are adsorbed onto the surface of the magnetic particles M by previously introducing the magnetic particles M into the adsorption chamber 120 filled with the nucleic acids and the adsorption solution, placing the lid 122 on the adsorption chamber 120, and shaking the adsorption chamber 120 by using an agitator or by hand(s) to vigorously mix the adsorption solution and the magnetic particles M in the adsorption chamber 120. The magnetic particle M herein is not specifically limited as long as it is a magnetic solid carrier that attracts objects and has a hydrophilic surface capable of attracting the nucleic acids, i.e., holding them via reversible physical adsorption in the presence of chaotropic ions. More specifically, the magnetic particle M is preferably a substance containing silicon dioxide such as silica, glass, and diatomite or chemically modified versions thereof. It is more preferable that the magnetic particle M is a complex with a magnetic substance or a superparamagnetic metal oxide.

After the magnetic particles M and the adsorption solution are mixed vigorously, the lid 122 is opened to connect the adsorption chamber 120 to the tube 100 or to pour the adsorption solution containing the magnetic particles M in the adsorption chamber 120 into the tube 100. In this event, the tube 100 is parallel to the direction of the gravity. Hereinafter, movement in the vertically downward direction of the magnetic force application unit 400 is referred to as falling, and movement in the vertically upward direction thereof (toward the adsorption chamber 120) is referred to as rising.

Sixth Plug and Seventh Plug The device for the extraction of nucleic acids according to this embodiment may include a sixth plug and a seventh plug in the tube 100.

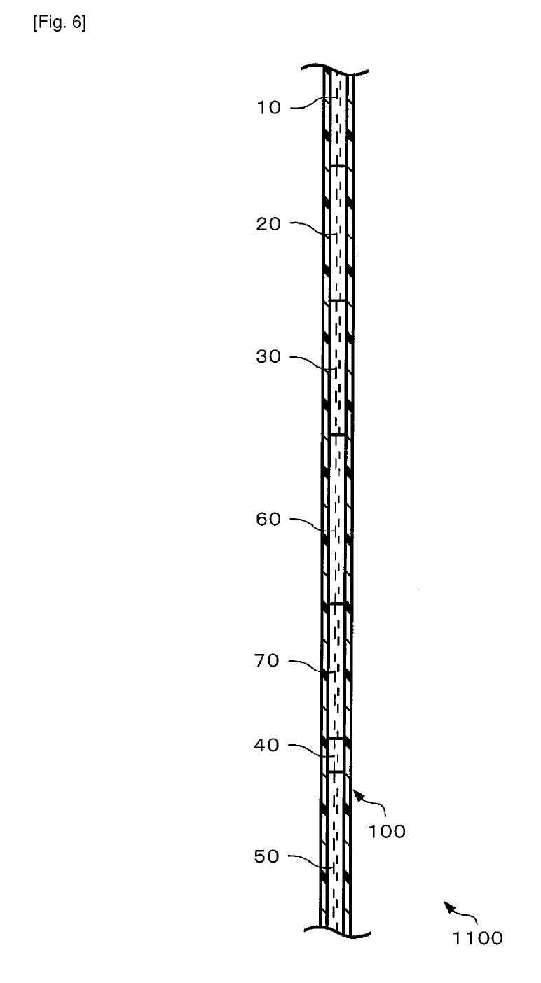

FIG. 6 is a view diagrammatically showing essential parts of the device for the extraction of nucleic acids of this modified version. More specifically, it is a view diagrammatically showing a device for the extraction of nucleic acids 1100 having a sixth plug 60 and a seventh plug 70 in the tube 100.

The device for the extraction of nucleic acids 1100 has the sixth plug 60 made of a second washing solution that is phase separated from the oil and the seventh plug 70 made of an oil, in this order from the third plug 30, that are added between the third plug 30 and the fourth plug 40 in the tube 100 of the aforementioned device for the extraction of nucleic acids.

The sixth plug 60 is provided adjacent to the third plug 30 in the tube 100 opposite to the second plug 20. The sixth plug 60 is made of the second washing solution. The second washing solution is a liquid that is phase separated from both the oil forming the third plug 30 and the oil forming the seventh plug 70. Examples of the second washing solution include water and a buffer having a solute at a concentration of 10 mM or lower, preferably 7 mM or lower, and more preferably, 5 mM or lower. The composition of the buffer is not specifically limited, and a tris-HCl buffer is an example. The buffer may contain EDTA (ethylenediaminetetraacetic acid). The second washing solution may be the same or different in composition from the first washing solution.

The volume of the sixth plug 60 is not specifically limited and can appropriately be determined based on, for example, the amount of the magnetic particles M carrying the nucleic acids adsorbed thereon. For example, when the volume of the magnetic particles M is equal to 0.5 uL, the volume of 10 uL or larger is enough for the sixth plug 60. It is preferable that the volume is equal to or larger than 20 uL but not larger than 50 uL, and more preferably, equal to or larger than 20 uL but not larger than 30 uL. With the volume of the sixth plug 60 within this range, the magnetic particles M can be washed sufficiently when the volume of the magnetic particles M is equal to 0.5 uL. Although a larger volume of the sixth plug 60 is preferable for washing the magnetic particles M, the volume can appropriately be determined in consideration of the length and the width of the tube 100 as well as the length of the sixth plug 60 depending thereon in the longitudinal direction of the tube 100.

The sixth plug 60 may be made up of two or more smaller plugs separated from each other by a plug of an oil. When the sixth plug 60 is made up of two or more plugs separated from the oil plug(s), this means that two or more plugs of the second washing solution are formed. With the sixth plug 60 separated by the oil plug(s), the concentration of a water-soluble substance (in a case where such water-soluble substance is subjected to washing) achieved in the plugs of the second washing solution is smaller than the concentration of the water-soluble substance achieved in the single plug of the first washing solution of the same volume. The sixth plug 60 may be separated into any number of smaller plugs. For example, when the sixth plug 60 is divided into two smaller plugs having an equal volume, the concentration of the water-soluble substance (in a case where such water-soluble substance is subjected to washing) can be reduced to ¼ of the concentration obtained without separating the sixth plug 60 by calculation. The number of the divisions of the sixth plug 60 is determined appropriately in consideration of, for example, the length of the tube 100 and the substance to be washed. When the first washing solution forming the second plug 20 and the second washing solution forming the sixth plug 60 are the same, a similar effect can be obtained to the one obtained by dividing the second plug 20 in the device for the extraction of nucleic acids that does not have the aforementioned sixth and seventh plugs 60 and 70.

The seventh plug 70 is made of an oil that is phase separated from both the sixth plug 60 and the elution solution forming the fourth plug 40 which are adjacent to the seventh plug 70. The oil forming the seventh plug 70 may be an oil that is different from the oil forming each of the first plug 10, the third plug 30, and the fifth plug 50. The oil may be similar to the one described in conjunction with the first plug 10.

Although the seventh plug 70 preferably contains neither air bubbles nor any other liquid, air bubbles and another liquid may be present as long as the magnetic particles M carrying the nucleic acids adsorbed thereon can pass through the seventh plug 70. In addition, it is preferable that no air or liquid is present between the seventh plug 70 and the adjacent fourth plug 40 and between the seventh plug 70 and the adjacent sixth plug 60. The air or another liquid may, however, be present as long as the magnetic particles M carrying the nucleic acids adsorbed thereon can pass through the tube 100. It is preferable that no air or liquid is present in the seventh plug 70.

The length of the seventh plug 70 in the longitudinal direction of the tube 100 is not specifically limited as long as the seventh plug 70 can be formed. The specific length of the seventh plug 70 in the longitudinal direction of the tube 100 is equal to or longer than 1 mm but not longer than 50 mm. The length is preferably equal to or longer than 1 mm but not longer than 30 mm, and more preferably, equal to or longer than 5 mm but not longer than 20 mm, in order to avoid that a travel distance of the magnetic particles M becomes too long. In the device for the extraction of nucleic acids 1100, when the length of the seventh plug 70 in the longitudinal direction of the tube 100 is long, the sixth plug 60 is less likely to be discharged in an aspect where the fourth plug 40 is discharged from the tube 100 on the side of the fifth plug 50. In this case, a specific length of the seventh plug 70 may be equal to or longer than 10 mm but not longer than 50 mm.

The seventh plug 70 serves to prevent the second washing solution forming the sixth plug 60 and the elution solution forming the fourth plug 40 from being mixed with each other. The seventh plug 70 may be made of an oil of a higher viscosity, which increases a “repellent effect” of the oil when the magnetic particles M are moved across the interface between the oil and the second washing solution forming the sixth plug 60. As a result, fewer water-soluble components on the surface of the magnetic particles M are brought into the seventh plug 70 (oil) when the magnetic particles are moved from the plug of the second washing solution forming the sixth plug 60 to the oil forming the seventh plug 70.

In the device for the extraction of nucleic acids 1100, the magnetic particles M carrying the nucleic acids adsorbed thereon can be washed in the second plug 20 and the sixth plug 60. This further improves an efficiency of washing the magnetic particles M.

In addition, in the device for the extraction of nucleic acids 1100, the first washing solution forming the second plug 20 may contain a chaotropic agent. For example, when guanidine hydrochloride is contained in the first washing solution forming the second plug 20, the magnetic particles M can be washed while the adsorption of the nucleic acids on the magnetic particles M is maintained or enhanced in the second plug 20. The concentration of the guanidine hydrochloride contained in the second plug 20 is, for example, equal to or higher than 3 mol/L but not higher than 10 mol/L, and preferably equal to or higher than 5 mol/L but not higher than 8 mol/L. With the concentration of the guanidine hydrochloride within this range, any foreign substances can be washed out while keeping stable adsorption of the nucleic acids on the magnetic particles M.

Furthermore, water or a buffer is used for the second washing solution forming the sixth plug 60. This allows washing with more stable adsorption of the nucleic acids on the magnetic particles M in the first washing solution forming the second plug 20. In addition, the magnetic particles M can further be washed while diluting the chaotropic agent in the second washing solution forming the sixth plug 60.

It will easily be appreciated that the device for the extraction of nucleic acids 1100 having the sixth plug 60 and the seventh plug 70 in the tube 100 can have the stopper and the adsorption chamber as described above as well as a liquid reservoir which will be described later, to achieve a similar effect to those described above.

3. Kit for the Extraction of Nucleic Acids

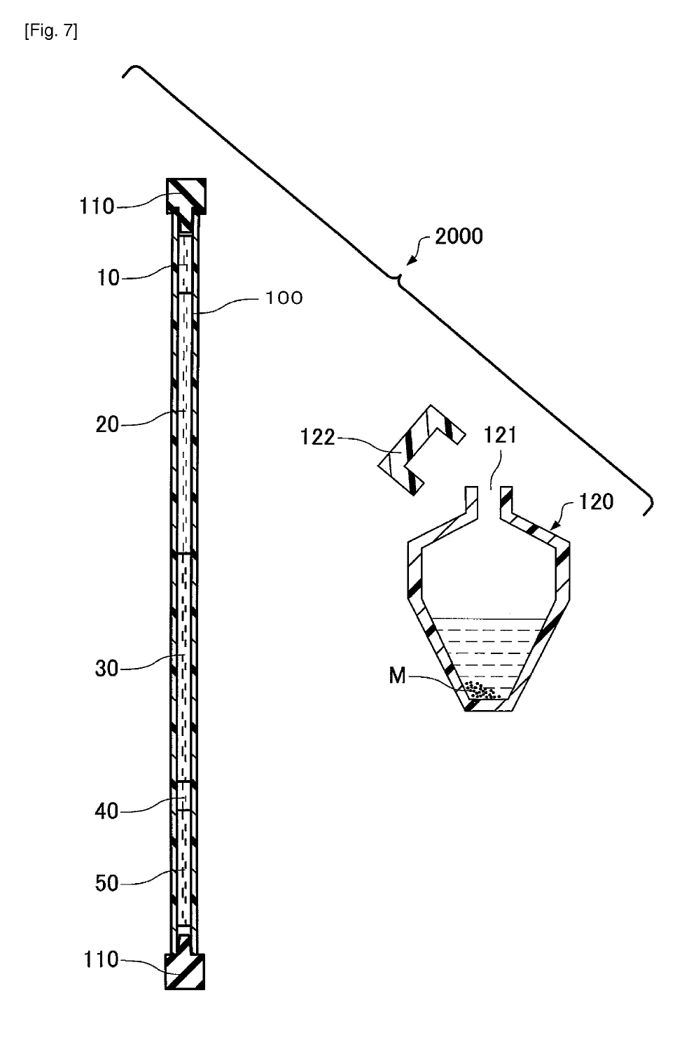

FIG. 7 is a view diagrammatically showing an example of a kit for the extraction of nucleic acids according to this embodiment. A kit for the extraction of nucleic acids 2000 shown in FIG. 7 includes the components that form an essential part of the aforementioned device for the extraction of nucleic acids 1000. Similar components that are described in conjunction with the section entitled “2. Device for the extraction of nucleic acids” are denoted by like reference numerals and detailed description thereof will be omitted.

The kit for the extraction of nucleic acids 2000 of this embodiment has the tube 100 and the adsorption chamber 120. The tube 100 contains the first plug 10 made of an oil, the second plug 20 made of the first washing solution that is phase separated from the oil, the third plug 30 made of an oil, the fourth plug 40 made of the elution solution that is phase separated from the oil, and the fifth plug 50 made of the oil, that are provided in this order. The adsorption chamber 120 can be connected to and communicated with the end of the tube 100 at the side of the first plug 10.

The tube 100 is similar to the tube 100 of the device for the extraction of nucleic acids 1000 with the ends thereof opened. The tube 100 has a hollow shape including a cavity formed therein through which a liquid can be passed in the longitudinal direction thereof. The inner shape, outer shape, size, characteristic, and material of the tube 100 are similar to those of the tube 100 of the device for the extraction of nucleic acids 1000. The plugs provided within the tube 100 are similar to those provided in the tube 100 of the device for the extraction of nucleic acids 1000. In addition, both ends of the tube 100 may be sealed with stoppers 110 that can freely be fitted to and removed from the tube 100. When the ends of the tube 100 are sealed with the stoppers 110, the kit for the extraction of nucleic acids 2000 can be stored and transferred more easily. Furthermore, with the stopper 110 sealing the end of the tube 100 at the side of the fifth plug 50, any displacement of the plugs in the tube 100 can be restricted while moving the magnetic particles M in the tube 100 during the use of the tube 100, which further facilitates the washing and the extraction. Moreover, the stopper 110 can freely be fitted to and removed from the tube 100, so that the end of the tube 100 at the side of the fifth plug 50 can be opened. Accordingly, it is easy to discharge the elution solution forming the fourth plug 40 into which the nucleic acids are eluted from the end of the tube 100 at the side of the fifth plug 50.

The adsorption chamber 120 is similar to the adsorption chamber 120 described in conjunction with the device for the extraction of nucleic acids 1000.

In the embodiment shown in FIG. 7, both ends of the tube 100 are sealed with the stoppers 110 that can freely be fitted to and removed from the tube 100. In addition, the kit for the extraction of nucleic acids 2000 may include the lid 122 capable of sealing the opening 121 in the adsorption chamber 120, which can freely be attached thereto and removed therefrom. The opening 121 in the adsorption chamber 120 may thus be sealed with the lid 122 that can freely be attached to and removed from the adsorption chamber 120. Furthermore, in the kit for the extraction of nucleic acids 2000, some or all of the components of the adsorption solution (liquid) may be contained in the adsorption chamber 120.

In the kit for the extraction of nucleic acids 2000, the adsorption chamber 120 may hold the adsorption solution and the magnetic particles M. This makes it possible to perform, in the adsorption chamber 120, a step of allowing the magnetic particles M to adsorb the nucleic acids in a sample (substance) after the sample is introduced into the adsorption chamber 120. No other vessel is thus required, so that a pre-treatment for PCR can be performed more quickly. In this case, the opening 121 in the adsorption chamber 120 may be sealed with the lid 122 that can freely be attached to and removed from the adsorption chamber 120, if necessary. The magnetic particles M will be described later.

By using an elastic material as a material of the adsorption chamber 120 as described above, the adsorption chamber 120 can be deformed and the inside of the tube 100 can be pressurized with the adsorption chamber 120 connected to the tube 100. In this way, a pressure can easily be applied to the tube 100 from the side of the first plug 10 in order to discharge the elution solution forming the fourth plug 40 containing the nucleic acids eluted therein from the end of the tube 100 at the side of the fifth plug 50. This configuration makes it possible to easily remove the elution solution into, for example, a reaction chamber for PCR.

The kit for the extraction of nucleic acids 2000 may include other components such as, for example, a stopper, a lid, an instruction manual, a reagent, and a case other than the tube 100 and the adsorption chamber 120. The illustrated embodiment describes an example where the tube 100 has five plugs therein. It is, however, readily understood that the tube 100 may have the sixth plug 60, the seventh plug 70 or other plug(s), if necessary, provided therein.

The kit for the extraction of nucleic acids 2000 of this embodiment has the adsorption chamber 120 that can be connected to and communicated with the end of the tube 100 at the side of the first plug 10. Accordingly, it is possible to allow the magnetic particles M to adsorb the nucleic acids by holding the magnetic particles M and the sample in the adsorption chamber 120. By connecting the adsorption chamber 120 to the end of the tube 100 at the side of the first plug 10, the magnetic particles M can easily be introduced into the tube 100 from the side of the first plug 10 in the tube 100. Furthermore, the kit for the extraction of nucleic acids 2000 of this embodiment has the adsorption chamber 120, so that the adsorption chamber 120 can be shaken to vigorously mix the liquid in the adsorption chamber 120. This allows the quick adsorption of the nucleic acids onto the magnetic particles M.

In addition, by connecting the adsorption chamber 120 to the tube 100, it is easy to introduce the magnetic particles M carrying the nucleic acids adsorbed thereon into the tube 100 through the first plug 10 and move them to the fourth plug 40. This allows the extraction of the nucleic acids in a significantly short period of time. The kit for the extraction of nucleic acids 2000 can be used to obtain the elution solution containing the nucleic acids in a highly pure form by moving the magnetic particles M carrying the nucleic acids adsorbed thereon through the tube 100. Accordingly, the kit for the extraction of nucleic acids 2000 can significantly reduce the time and labor required for the pre-treatment for PCR.

4. Method for the Extraction of Nucleic Acids

The apparatus for the extraction of nucleic acids 3000, the kit for the extraction of nucleic acids 2000, and the device for the extraction of nucleic acids 1000, which are described above, and their variations which will be described later can all suitably be used for a method for the extraction of nucleic acids as a method of manipulating the solid carriers of this embodiment.

A method using the aforementioned kit for the extraction of nucleic acids 2000 is described as an example of the method for the extraction of nucleic acids of this embodiment.

The method for the extraction of nucleic acids of this embodiment comprises the steps of: introducing a sample containing the nucleic acids into the flexible adsorption chamber 120 in which the magnetic particles M and the adsorption solution are contained; shaking the adsorption chamber 120 to allow the magnetic particles M to adsorb the nucleic acids; connecting the adsorption chamber 120 to the end of the tube 100 at the side of the first plug 10 so that the adsorption chamber 120 is communicated with the tube 100, the tube 100 having the first plug 10 made of an oil, the second plug 20 made of the first washing solution that is phase separated from the oil, the third plug 30 made of an oil, the fourth plug 40 made of the elution solution that is phase separated from the oil, and the fifth plug 50 made of an oil provided therein in this order; applying a magnetic force to move the magnetic particles M from the inside of the adsorption chamber 120 to the position of the fifth plug 50 through the tube 100; and eluting the nucleic acids from the magnetic particles M into the elution solution forming the fourth plug.

In the method for the extraction of nucleic acids of this embodiment, various particles (e.g., silica particles, polymer particles, and particles of a magnetic substance) may be used as long as they are magnetic particles capable of adsorbing nucleic acids in the adsorption solution and can be moved in the tube 100 using a magnetic force. In an embodiment of the method for the extraction of nucleic acids described below, such magnetic particles are used as the magnetic particles M that contain a magnetic substance and can adsorb nucleic acids on the surface thereof. When particles other than the magnetic particles are moved in the tube, the gravity or a potential difference may also be used.

In the method for the extraction of nucleic acids of this embodiment, a material that transmits the magnetic force is selected for the adsorption chamber 120 and the tube 100. The magnetic force is applied to the adsorption chamber 120 and the tube 100 from the outside to move the magnetic particles M within the adsorption chamber 120 and the tube 100.

The sample contains the target nucleic acids. Hereinafter, this may merely be referred to as the target nucleic acids. The target nucleic acids may be, for example, DNAs or RNAs (DNA is a deoxyribonucleic acid, and/or RNA is a ribonucleic acid). The target nucleic acids are extracted from the sample and eluted into the elution solution using the method for the extraction of nucleic acids of this embodiment, and then used as, for example, a template for PCR. Examples of the sample include blood, nasal mucus, oral mucus, and various other biological samples.

4.1. Step of Introducing the Sample into the Vessel

The step of introducing a sample into the adsorption chamber 120 may be performed by, for example, taking the sample with a cotton swab, inserting the cotton swab into the adsorption chamber 120 through the opening 121, and dipping it in the adsorption solution. The sample may be introduced into the adsorption chamber 120 through the opening 121 using, for example, a pipette. When the sample is a paste or a solid material, it may be put into the adsorption chamber 120 or adhered to the inner surface of the adsorption chamber 120 using, for example, a spoon or tweezers inserted through the opening 121.

4.2. Step of Allowing the Magnetic Particles to Adsorb the Nucleic Acids

The step of allowing the magnetic particles to adsorb the nucleic acids is performed while shaking the adsorption chamber 120. This step can be performed more efficiently by using the lid 122, if used for sealing the opening 121 in the adsorption chamber 120, to seal the adsorption chamber 120. During this step, the target nucleic acids are adsorbed onto the surface of the magnetic particles M due to the effect of the chaotropic agent. Proteins or nucleic acids other than the target nucleic acids may also be adsorbed onto the surface of the magnetic particles M in this step.

The shaking of the adsorption chamber 120 may be achieved by using an apparatus such as a vortex shaker. Alternatively, the adsorption chamber 120 may manually be shaken by an operator using his or her hand(s). In addition, the adsorption chamber 120 may be shaken while externally applying a magnetic force taking advantage of the magnetism of the magnetic particles M. The time duration during which the adsorption chamber 120 is shaken can appropriately be determined. For example, when an approximate shape of the adsorption chamber 120 is a hollow cylindrical shape having a diameter of about 20 mm and a height of about 30 mm, the content of the adsorption chamber 120 is mixed vigorously by shaking the adsorption chamber 120 by hands for 10 seconds and the nucleic acids are adsorbed onto the surface of the magnetic particles M.

4.3. Step of Connecting the Vessel to the Tube

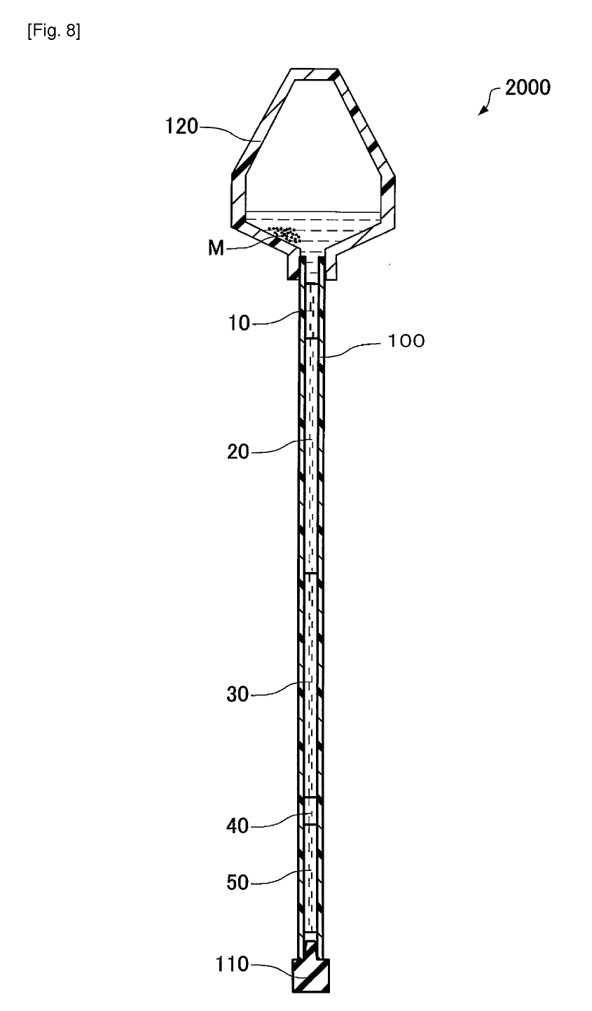

FIG. 8 is a view diagrammatically showing an example of a kit for the extraction of nucleic acids according to this embodiment.

As shown in FIG. 8, the adsorption chamber 120 is connected to the end of the tube 100 at the side of the first plug 10. The plugs in the tube 100 are less likely to move in the tube even after the stopper 110 is removed from the end at the side of the first plug 10 because the stopper 110 is left at the end at the side of the fifth plug 50. This step is performed after the stopper 110 at the end of the tube 100 at the side of the first plug 10, if any, is removed. The adsorption chamber 120 and the tube 100 are connected so that the content thereof does not escape therefrom. The adsorption chamber 120 is communicated with the tube 100 so that the content can flow between them.

4.4. Step of Moving the Magnetic Particles

After the aforementioned step, the magnetic particles M carrying the nucleic acids adsorbed thereon in the adsorption chamber 120 are ready to be introduced into the tube 100. In the liquids contained in the tube 100, the magnetic particles M having the magnetism that are capable of carrying the nucleic acids are manipulated. A method of introducing the magnetic particles M carrying the nucleic acids adsorbed thereon into the tube 100 is not specifically limited and, for example, the gravity or a centrifugal force may be used. This embodiment, however, uses the external application of a magnetic force to the adsorption chamber 120 and the tube 100. The magnetic force may be applied by using, for example, a permanent magnet or an electromagnet. It is preferable that a permanent magnet is used for this application purpose because less heat is generated. When permanent magnets are used, the magnets may be moved by an operator with his or her hand(s) or, alternatively, by a mechanical device. The magnetic particles M are attracted by magnetic force, so that this property is used to alter the relative position between the adsorption chamber 120 plus the tube 100 and the permanent magnets to move the magnetic particles M from the adsorption chamber 120 to the tube 100. The magnetic particles M are manipulated using the magnetic force application unit 300 adapted to applying a magnetic force to the magnetic particles M in the tube 100 so that the relative position between the magnetic force application unit 400 and the magnetic particles M is altered over a period of time by means of directing the magnetic particles M in a direction along either one or a combination of the vectors of the longitudinal direction of the tube 100 and the direction crossing the longitudinal direction of the tube 100. As a result, the magnetic particles M are moved sequentially from the first plug 10 to the fourth plug 40. The resident time of the magnetic particles M in each plug when they pass through that plug is not specifically limited. In addition, the magnetic particles M may be rockd within a single plug in the longitudinal direction of the tube 100.

Now, an example of a method of externally applying a magnetic force is described in which the magnetic particles M are moved by applying a magnetic force to the adsorption chamber 120 and the tube 100 of this embodiment from the outside.

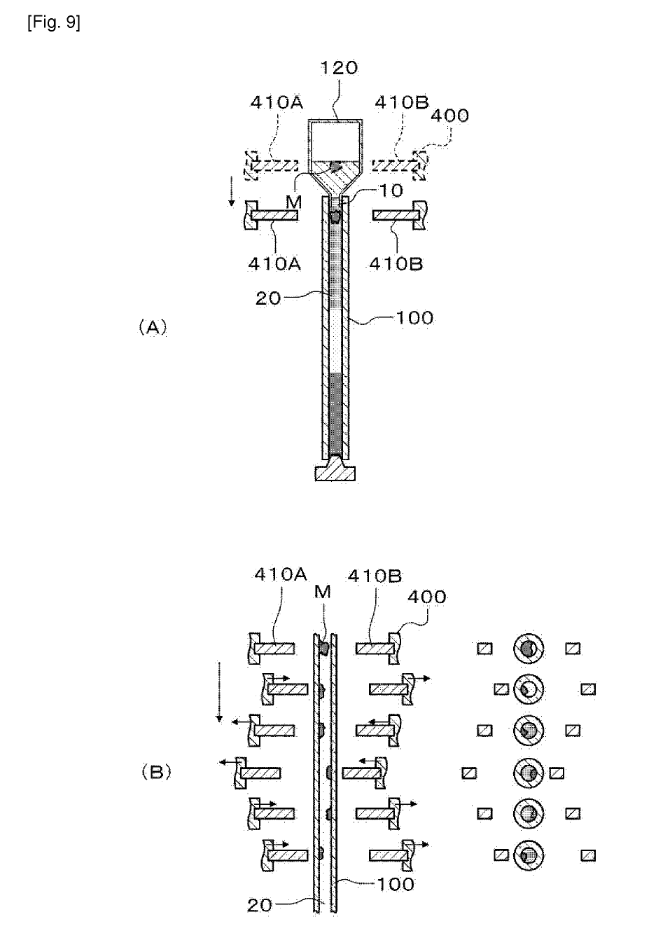

FIGS. 9 to 11 are views showing a method of externally applying a magnetic force to move the magnetic particles M according to this embodiment. The magnetic force application unit 400 comprises, as shown in FIG. 9(A), a pair of permanent magnets 410A, 410B opposed to each other at a certain distance. The permanent magnets 410A, 410B are faced to each other with the tube 100 placed between them after the tube 100 is mounted on the tube mount 300 (see FIG. 1).

First, in order to introduce the magnetic particles M in the adsorption chamber 120 into the tube 100, the magnetic force application unit 400 is lowered from the level of the adsorption chamber 120 until the pair of permanent magnets 410A, 410B of the magnetic force application unit 400 reaches the level of the second plug 20 in the tube 100.

Next, in order to wash the magnetic particles M, the magnetic force application unit 400 is lowered in the longitudinal direction of the tube 100 while repeatedly moving the permanent magnets in a horizontal direction, as shown in FIG. 9(B). More specifically, the permanent magnets are moved so that one permanent magnet 410A (or the permanent magnet 410B) is moved closer to the side of the tube 100 while the other permanent magnet 410B (or the permanent magnet 410A) is moved away from the side of the tube 100, and then vise versa so that the relative magnitudes of the magnetic force applied to the tube 100 by the permanent magnets 410A, 410B is alternated. It is preferable that the washing of the magnetic particles M by the first washing solution forming the second plug 20 is performed while passing the magnetic particles M through the first washing solution forming the second plug 20 over a long distance. With the present method, the magnetic particles M can travel in the liquid over a distance that is enough to achieve the effect of washing the surface of the magnetic particles M with the first washing solution forming the second plug 20 in the tube 100 by falling the magnetic particles M in the longitudinal direction of the tube 100 while moving them laterally.

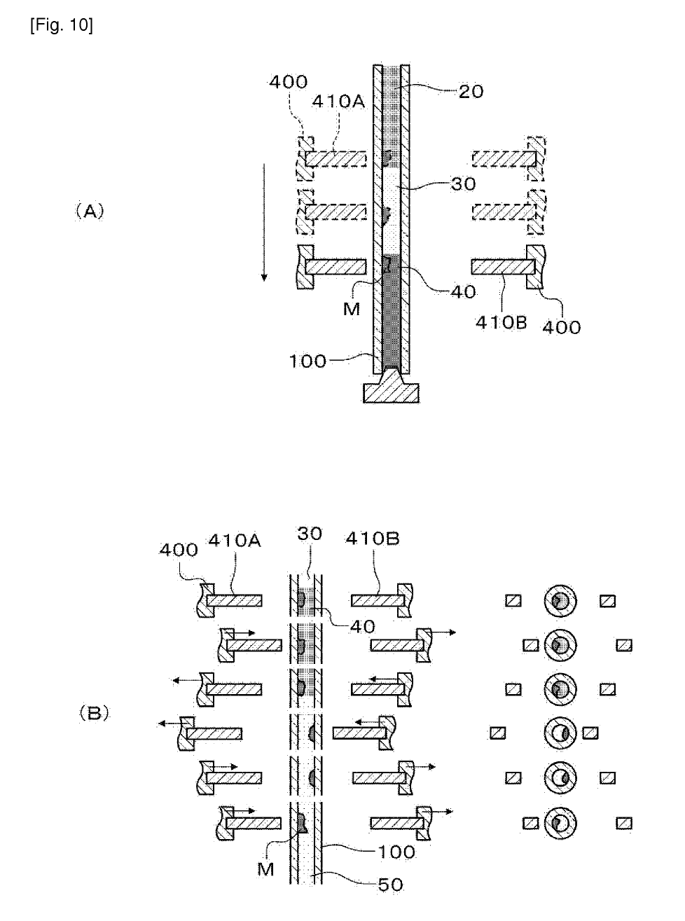

Next, in order to move the magnetic particles M from the second plug 20 to the third plug 30, one permanent magnet 410A (or the permanent magnet 410B) of the magnetic force application unit 400 is moved closer to the side of the tube 100, and then the magnetic force application unit 400 is lowered to the level of the third plug 30, as shown in FIG. 10(A). Since the magnetic force is larger on a portion of the side of the tube 100 because of the permanent magnet closer to the tube, the magnetic particles M are gathered or collected at that position on the side of the tube 100. These gathered magnetic particles M can be moved to the third plug without being captured at the interface between the first washing solution forming the second plug 20 and the oil forming the third plug 30.

Next, in order to move the magnetic particles M from the third plug 30 to the fourth plug 40, the magnetic force application unit 400 is lowered until the permanent magnet 410A (or the permanent magnet 410B) of the magnetic force application unit 400 reaches the level of the fourth plug 40 while keeping one permanent magnet 410A (or the permanent magnet 410B) closer to the side of the tube 100 as shown in FIG. 10(A). The magnetic particles M can thus be moved from the third plug 30 to the fourth plug 40.

Subsequently, as shown in FIG. 10(B), one permanent magnet 410A (or the permanent magnet 410B) is moved closer to the side of the tube 100 while the other permanent magnet 410B (or the permanent magnet 410A) is moved away from the side of the tube 100, and then vise versa, to alternate the relative magnitudes of the magnetic force applied to the tube 100 by the permanent magnets 410A, 410B when the magnetic particles M are present in the fourth plug 40. In other words, the magnetic particles M are displaced in the fourth plug 40 at least between two different points in the direction perpendicular to the longitudinal direction of the tube 100. As a result, the nucleic acids adsorbed on the magnetic particles M can be eluted in the elution solution forming the fourth plug 40. In addition to the aforementioned lateral movement of the magnetic force application unit 400, it may be moved vertically in the longitudinal direction of the tube 100. This ensures the magnetic particles M to reside in the fourth plug 40.

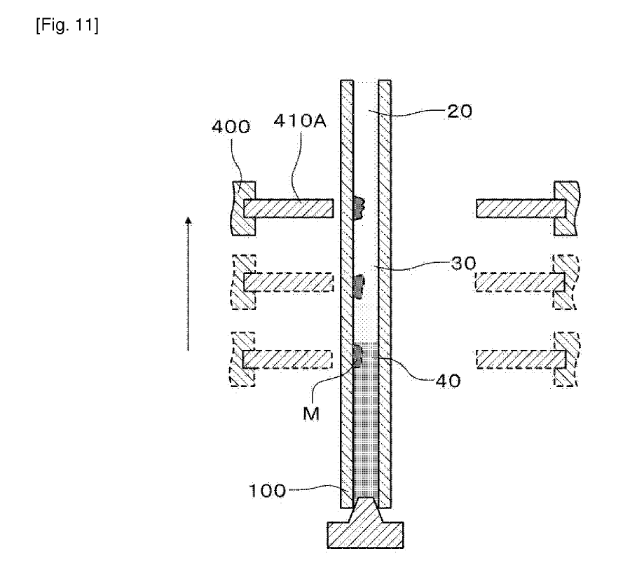

After the magnetic particles M are rockd enough in the fourth plug 40, one permanent magnet 410A (or the permanent magnet 410B) of the magnetic force application unit 400 is moved closer to the side of the tube 100, and the permanent magnets 410A and 410B are moved upward the permanent magnet 410A (or the permanent magnet 410B) of the magnetic force application unit 400 reaches the level of the second plug 20, as shown in FIG. 11. The magnetic force is larger on a portion of the side of the tube 100 and the magnetic particles M are thus gathered on the inner surface of the tube 100 near the permanent magnet 410A (or the permanent magnet 410B). Accordingly, the magnetic particles M can be moved to the second plug 20 without being captured at the interface between the elution solution forming the fourth plug 40 and the oil forming the third plug and at the interface between the first washing solution forming the second plug 20 and the oil forming the third plug 30.

Subsequently, the elution solution forming the fourth plug 40 containing the nucleic acids eluted therein is dispensed from the tube 100 or directly introduced into a reaction chamber for PCR. In this way, the intended nucleic acids are extracted from the sample for a PCR reaction.

4.5. Step of Eluting the Nucleic Acids

After the magnetic particles M reaches the fourth plug 40, the nucleic acids carried on the magnetic particles M are eluted into the elution solution forming the fourth plug 40 due to the action of the elution solution. During this step, the nucleic acids are eluted from the sample into the elution solution. The nucleic acids are thus extracted from the sample.

4.6. Step of Discharging the Fourth Plug from the Tube

The method for the extraction of nucleic acids of this embodiment may include a step of discharging the fifth plug 50 and the fourth plug 40 from the end of the tube 100 opposite to the end connected to the adsorption chamber 120 by deforming the adsorption chamber.

This step can be performed by deforming the adsorption chamber 120 after the “4.5. Step of eluting the nucleic acids.” The fifth plug 50 is discharged first before the extrusion of the fourth plug 40. It should be noted that the stopper 110 sealing the end of the tube 100 at the side of the fifth plug 50 is removed prior to this step to open the end of the tube 100 at the side of the fifth plug 50.

When an external force is applied to the adsorption chamber 120 to deform it by the increase of the inner pressure, the plugs are pushed down and moved due to the pressure in the direction from the first plug 10 to the fifth plug 50 in the tube 100. As a result, the fifth plug 50 and the fourth plug 40 are discharged in this order through the end of the tube 100 at the side of the fifth plug 50. The third plug 30 (or the seventh plug 70) may also be discharged, but the second plug 20 (or the sixth plug 60) is prevented from being discharged. In this case, for example, it is easy to prevent the second plug 20 (or the sixth plug 60) from being discharged when the volume of the third plug 30 (or the seventh plug 70) is larger than the volume of other plugs, and the third plug 30 (or the seventh plug 70) is long enough in the longitudinal direction of the tube 100.

The fourth plug 40 and the fifth plug 50 are discharged into, for example, a reaction chamber for PCR. The elution solution and the oil are thus removed into the reaction chamber for PCR, but the oil does not usually affect reactions of PCR. Accordingly, an oil similar to the oil forming the fifth plug 50 may previously be contained in the reaction chamber for PCR. In this case, the present step may be performed with the end of the tube 100 dipped in the oil. This allows the introduction of the elution solution containing the target nucleic acids into the reaction chamber for PCR without exposing the solution to the outside air. When the method for the extraction of nucleic acids of this embodiment includes the present step, the elution solution containing the target nucleic acids can easily be removed into, for example, the reaction chamber for PCR.

4.7. Effects

In the method for the extraction of nucleic acids of this embodiment, extraction of the nucleic acids can easily be performed in a significantly short period of time. Using the method for the extraction of nucleic acids of this embodiment, it is possible to obtain the elution solution containing the nucleic acids in a highly pure form by moving the magnetic particles M carrying the nucleic acids adsorbed thereon through the tube 100. Accordingly, the method for the extraction of nucleic acids of this embodiment can significantly reduce the time and labor required for the pre-treatment for PCR.

5. Experiments

Experiments are now described and the present invention is described more in detail below, but the present invention is not limited to these experiments.

5.1. Experiment 1

In the Experiment 1, the aforementioned kit for the extraction of nucleic acids 2000 was used with the first plug 10 through the seventh plug 70 provided in the tube 100.

First, a suspension of 375 uL of the adsorption solution and uL of the magnetic beads (the magnetic particles M) was contained in a polyethylene vessel (the adsorption chamber 120) having a volume of 3 mL. The composition of the adsorption solution was an aqueous solution of 76 wt. % of guanidine hydrochloride, 1.7 wt. % of disodium dihydrogen ethylenediamine tetraacetate dihydrate, and 10 wt. % of polyoxyethylene sorbitan monolaurate (TOYOBO, MagExtractor-Genome-, NPK-1). In addition, a suspension containing 50 vol. % of magnetic silica particles and 20 wt. % of lithium chloride was used as a suspension of magnetic beads.

Using a pipette, 50 uL of the blood collected from a human subject was added to the adsorption chamber 120 through the opening therein. The adsorption chamber 120 was fitted with the lid 122 and the content was mixed vigorously by hands for 30 seconds. Thereafter, the lid 122 of the adsorption chamber 120 was removed and the adsorption chamber 120 was connected to the tube 100. The tube 100 had the stoppers 110 at both ends thereof, and the stopper 110 at the end closer to the first plug 10 was removed before connecting the adsorption chamber 120 to the tube 100.

A silicone oil was used as the first, third, seventh, and fifth plugs 10, 30, 70, and 50. An aqueous solution of 76 wt. % of guanidine hydrochloride was used as the first washing solution of the second plug 20. In addition, a tris HCl buffer (solute concentration of 5 mM) of pH 8.0 was used as the second washing solution of the sixth plug 60. Sterilized water was used as the elution solution of the fourth plug 40.

The permanent magnets 410 were moved by hands and the magnetic particles M in the adsorption chamber 120 were introduced into the tube 100. Then, the magnetic particles M were moved to the fourth plug 40. The magnetic particles M were present in the plugs in the tube 100 for approximately the following time durations: 3 seconds each in the first, third, and seventh plugs 10, 30, and 70, 20 seconds in the second plug 20, 20 seconds in the sixth plug 60, and 30 seconds in the fourth plug 40. In the second plug 20 and the sixth plug 60, the magnetic particles M were not moved laterally. In addition, the volumes of the second plug 20, the sixth plug 60, and the fourth plug 40 were 25 uL, 25 uL, and 1 uL, respectively.

Next, the stopper at the end of the tube 100 at the side of the fifth plug 50 was removed, and the vessel was deformed by hands to discharge the fifth plug 50 and the fourth plug 40 into a reaction chamber for PCR. This operation was performed after the magnetic particles M were moved using the permanent magnets 410 back to the second plug 20.

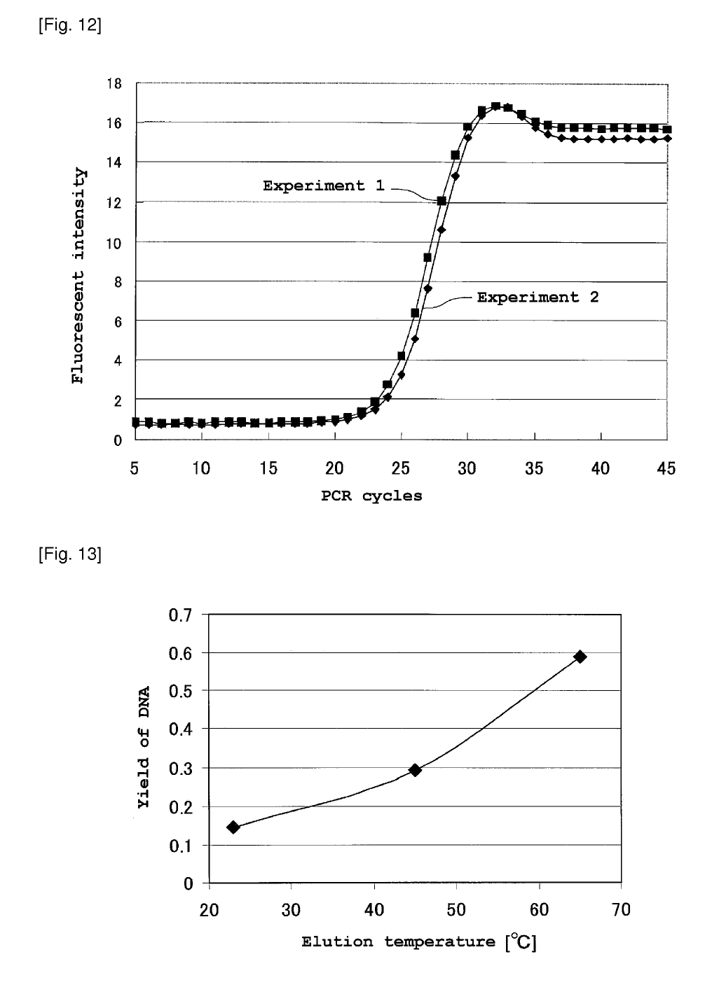

Subsequently, 19 uL of PCR reagent was added to the elution solution to perform real-time PCR according to an ordinary method. The PCR reagent used was a mixture of: 4 uL of LightCycler 480 Genotyping master (Roche Diagnostics 4 707 524), 0.4 uL of SYBR Green I (S7563 available from Life Technologies) diluted to 1/1000 with sterilized water, 0.06 uL of 100 uM beta-actine primers (F/R), and 14.48 uL of sterilized water. A PCR amplification curve in the Experiment 1 is shown in FIG. 12.

FIG. 12 is a graph showing the results obtained in this experiment. The ordinate in FIG. 12 represents the fluorescent intensity while the abscissa represents PCR cycles.

5.2. Experiment 2

In the Experiment 2, the nucleic acids were extracted using an ordinary method of extracting nucleic acids.

First, a suspension of 375 uL of the adsorption solution, and uL of the magnetic beads (the magnetic particles M) was contained in a polyethylene vessel (the adsorption chamber 120) having a volume of 1.5 mL. The composition of the suspension of the adsorption solution and the magnetic beads was similar to the one described in the aforementioned experiment.

Using a pipette, 50 uL of the blood collected from a human subject was added to the adsorption chamber 120 through the opening therein. The adsorption chamber 120 was fitted with the lid 122 and the content was mixed vigorously for 10 minutes using a vortex mixer. B/F separation was then performed using the magnetic stand and a pipette. In this state, the magnetic particles M and a small amount of the adsorption solution were left in the adsorption chamber 120.

Next, 450 uL of the first washing solution having the same composition as in the Experiment 1 was introduced into the adsorption chamber 120, and the lid 122 was fitted to the adsorption chamber 120. The content was mixed vigorously for 5 seconds using a vortex mixer. The first washing solution was removed using the magnetic stand and the pipette. This operation was repeated twice. In this state, the magnetic particles M and a small amount of the first washing solution were left in the adsorption chamber 120.

Next, 450 uL of the second washing solution having the same composition as in the Experiment 1 was introduced into the adsorption chamber 120, and the lid 122 was fitted to the adsorption chamber 120. The content was mixed vigorously for 5 seconds using a vortex mixer. The second washing solution was removed using the magnetic stand and the pipette. This operation was repeated twice. In this state, the magnetic particles M and a small amount of the second washing solution were left in the adsorption chamber 120.

Furthermore, 50 uL of sterilized water (the elution solution) was added to the adsorption chamber 120. The adsorption chamber 120 was fitted with the lid 122 and the content was mixed vigorously for 10 minutes using a vortex mixer. A supernatant was collected using the magnetic stand and the pipette. This supernatant contains the target nucleic acids.

Subsequently, 1 uL of the elution solution was removed into a new tube and 19 uL of PCR reagent was added to the elution solution to perform real-time PCR according to an ordinary method. The PCR reagent used was a mixture of: 4 uL of LightCycler 480 Genotyping master (Roche Diagnostics 4 707 524), 0.4 uL of SYBR Green I (S7563 available from Life Technologies) diluted to 1/1000 with sterilized water, 0.06 uL of 100 uM beta-actine primers (F/R), and 14.48 uL of sterilized water. A PCR amplification curve in this experiment is shown in FIG. 12.

5.3. Experiment 3

In the Experiment 3, the aforementioned kit for the extraction of nucleic acids 2000 was used with the first plug 10 through the fifth plug 50 provided in the tube 100.

The composition of the suspension of the adsorption solution and the magnetic beads was similar to those used in the Experiment 1, and a silicone oil was used as the first, third, and fifth plugs 10, 30, and 50 as in the case of the Experiment 1.

A tris-HCl buffer (solute concentration of 5 mM) of pH 8.0 was used as the first washing solution of the second plug 20. Sterilized water was used as the elution solution of the fourth plug 40.

Using a pipette, 50 uL of the blood collected from a human subject was added to the adsorption chamber 120 through the opening therein. The adsorption chamber 120 was fitted with lid 122 and the content was mixed vigorously by hands for 30 seconds. Thereafter, the lid 122 of the adsorption chamber 120 was removed and the adsorption chamber 120 was connected to the tube 100. The tube 100 had the stoppers 110 at both ends thereof, and the stopper 110 at the end closer to the first plug 10 was removed before connecting the adsorption chamber 120 to the tube 100.

The permanent magnets 410 were moved by hands and the magnetic particles M in the adsorption chamber 120 were introduced into the tube 100. Then, the magnetic particles M were moved to the fourth plug 40. The magnetic particles M were present in the plugs in the tube 100 for approximately the following time durations: 3 seconds each in the first and third plugs 10 and 30, seconds in the second plug 20, and 30 seconds in the fourth plug 40. In the second plug 20, the magnetic particles M were not moved laterally. In addition, the volumes of the second plug 20 and the fourth plug 40 were 25 uL and 1 uL, respectively.

Next, the stopper 110 at the end of the tube 100 at the side of the fifth plug 50 was removed, and the adsorption chamber 120 was deformed by hands to discharge the fifth plug 50 and the fourth plug 40 into a reaction chamber for PCR. This operation was performed after the magnetic particles M were moved using the permanent magnets 410 back to the second plug 20.

Subsequently, 19 uL of PCR reagent was added to the elution solution to perform real-time PCR according to an ordinary method. The PCR reagent used was a mixture of: 4 uL of LightCycler 480 Genotyping master (Roche Diagnostics 4 707 524), 0.4 uL of SYBR Green I (S7563 available from Life Technologies) diluted to 1/1000 with sterilized water, 0.06 uL of 100 uM beta-actine primers (F/R), and 14.48 uL of sterilized water.

The amplification curves were similar to those shown in FIG. 12. In this experiment, when a similar experiment was performed using 76 wt. % of guanidine hydrochloride as the first washing solution of the second plug 20, a delay of the rising edge corresponding to 10 cycles or more was observed as compared to the amplification curve obtained in the Experiment 1.

5.4. Experiment 4

Effect of Elution Temperature on the Yield of DNAs

In the Experiment 4, the nucleic acids were extracted using an ordinary method of extracting nucleic acids.

First, a suspension of 375 uL of the adsorption solution, and uL of the magnetic beads (the magnetic particles M) was contained in a polyethylene vessel (the adsorption chamber 120) having a volume of 1.5 mL. The composition of the suspension of the adsorption solution and the magnetic beads was similar to the one described in the aforementioned experiment.

Next, using a pipette, 50 uL of the genomic DNA solution adjusted to 1 ng/uL was added to the adsorption chamber 120 through the opening therein. The adsorption chamber 120 was fitted with the lid 122 and the content was mixed vigorously for minutes using a vortex mixer. B/F separation was then performed using the magnetic stand and the pipette. In this state, the magnetic particles M and a small amount of the adsorption solution were left in the adsorption chamber 120.

Next, 450 uL of the first washing solution having the same composition as in the Experiment 1 was introduced into the adsorption chamber 120, and the lid 122 was fitted to the adsorption chamber 120. The content was mixed vigorously for 5 seconds using a vortex mixer. The first washing solution was removed using the magnetic stand and the pipette. This operation was repeated twice. In this state, the magnetic particles M and a small amount of the first washing solution were left in the adsorption chamber 120.

Next, 450 uL of the second washing solution having the same composition as in the Experiment 1 was introduced into the adsorption chamber 120, and the lid 122 was fitted to the adsorption chamber 120. The content was mixed vigorously for 5 seconds using a vortex mixer. The second washing solution was removed using the magnetic stand and the pipette. This operation was repeated twice. In this state, the magnetic particles M and a small amount of the second washing solution were left in the adsorption chamber 120.

Furthermore, 50 uL of sterilized water (the elution solution) was added to the adsorption chamber 120. The adsorption chamber 120 was fitted with the lid 122 and the content was mixed vigorously for 5 seconds using a vortex mixer, which was heated for 2 minutes using a tube heater. Thereafter, the content was mixed vigorously for another 10 seconds using the vortex mixer. A supernatant was collected using the magnetic stand and the pipette. Three different temperatures were used to heat the tube heater: 23° C. (allowed to stand at a room temperature), 45° C., and 65° C.

Subsequently, 1 uL of the elution solution was removed into a new tube and 19 uL of PCR reagent was added to the elution solution to perform real-time PCR according to an ordinary method. As a comparative sample, a genomic DNA solution adjusted to 1 ng/uL was also added to the PCR sample. The PCR reagent used was a mixture of: 4 uL of LightCycler 480 Genotyping master (Roche Diagnostics 4 707 524), 0.4 uL of SYBR Green I (S7563 available from Life Technologies) diluted to 1/1000 with sterilized water, 0.06 uL of 100 uM beta-actine primers (F/R), and 14.48 uL of sterilized water.

The relationship between the elution temperature and the yield of DNAs is shown in FIG. 13.

FIG. 13 is a graph showing the relationship between the elution temperature and the yield of DNAs in this embodiment. This result was calculated from the cycle of the rising edge of the real-time PCR. The yield of DNAs can be given as a ratio relative to the comparative sample (corresponding to 1) by the equation 2 (Ct0-Ct1), in which Ct0 represents cycle of the rising edges for the comparative sample and Ct1 represents a cycle of the rising edges for the extraction sample.

5.5. Results of the Experiments

The following things were found from the aforementioned experiments.

(1) When the time duration required for the extraction of the nucleic acid, that is a pre-treatment of PCR, was compared, the time after the insertion of the sample into the adsorption chamber 120 and before the introduction of the target nucleic acids into the reaction chamber for PCR was approximately 2 minutes in the Experiment 1. It was approximately 30 minutes in the Experiment 2. From these results, it was found that the time required for the extraction of the nucleic acids was significantly shorter in the method for the extraction of nucleic acids of the Experiment 1 than in the method for the extraction of nucleic acids of the Experiment 2.

(2) In addition, the amount of the washing solution in the Experiment 1 was 1/18 of the amount in the Experiment 2. Furthermore, the amount of the elution solution in the Experiment 1 was about 1/50 of the amount in the Experiment 2. It was thus found that the Experiment 1 was significantly smaller in the amounts of the washing solution and the elution solution as compared to the Experiment 2.

(3) Moreover, when the concentration of the target nucleic acids in the elution solution is compared under the assumption that the amounts of the adsorption solution and the elution solution are the same, the concentration is ideally 50 times higher in the Experiment 1 than in the Experiment 2. In the experiments described above, however, the blood sample contains a large amount of the nucleic acids, which exceeds the maximum amount that can be adsorbed onto 1 uL of the magnetic particles M. Accordingly, not all the nucleic acids contained in the blood sample can be collected, so the concentration in the Experiment 1 is not 50 times higher than that in the Experiment 2. For such samples that contain a small amount of the nucleic acids and these nucleic acids can all be adsorbed onto 1 uL of the magnetic particles M, the concentration in the Experiment 1 will be 50 times higher than that in the Experiment 2.

(4) From the graph shown in FIG. 12, it was found that the rising edge of the amplification curve for the nucleic acids was observed about 0.6 cycle earlier in the Experiment 1 than in the Experiment 2 even in the whole blood sample containing a large amount of the nucleic acids. In other words, it was found that the reaction solution for PCR used in the Experiment 1 contained the target nucleic acids at a higher concentration than in the reaction solution for PCR used in the Experiment 2. This confirmed that the concentration of the target nucleic acids in the elution solution was higher in the Experiment 1 than in the Experiment 2.

(5) From the result in the Experiment 3, it was found that nucleic acids could be extracted enough even if the second plug 20 contains a buffer. When the guanidine solution was used as the second plug 20, it was found that a significant delay of the rising edge of the PCR amplification curve occurred due to the inhibition of the enzyme reaction. In addition, it was also found that the effect of the inhibition of the enzyme reaction in the guanidine solution could be reduced by diluting the elusion solution by a factor of at least 1000.

(6) From the result in the Experiment 4, it was found that the yield of DNAs could be large enough for use in PCR when the temperature of the fourth plug 40 was around 40° C. or higher.

### Embodiment 2

A method of externally applying a magnetic force that is different from that described in the Embodiment 1 is described according to the method of manipulation of the present invention.

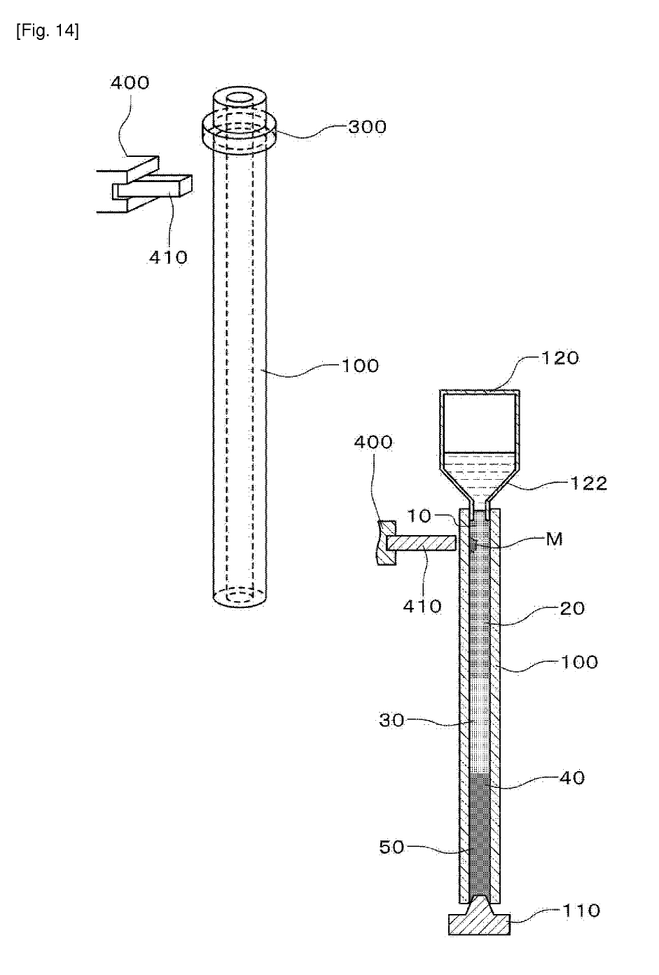

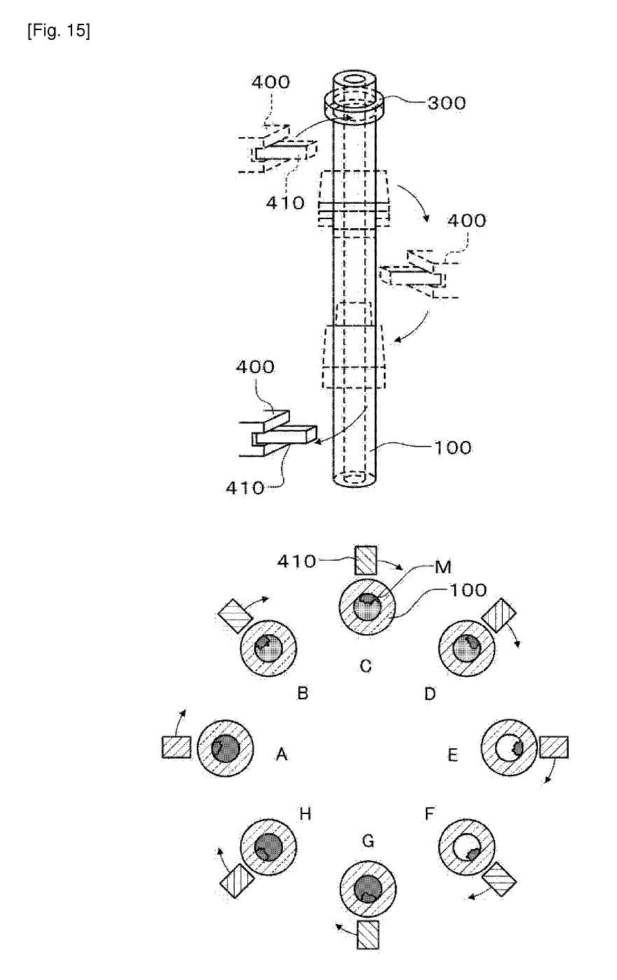

FIGS. 14 and 15 are views showing an exemplified configuration of the permanent magnet 410 and the tube 100 according to this embodiment. FIG. 14 shows a structure and FIG. 15 shows a moving procedure.

The permanent magnet 410 is connected to the magnetic force application unit 400. Either one or both of the permanent magnet 410 and the tube 100 are connected to a moving mechanism that can change the relative position between them. As shown in the Embodiment 1, as a method of introducing the magnetic particles M carrying the nucleic acids adsorbed thereon into the tube 100 having the first washing solution forming the second plug 20 and the elution solution forming the fourth plug 40 that are filled as the plugs separated by the oil forming the third plug 30, and moving them in the tube 100, the permanent magnet 410 of the magnetic force application unit 400 having this single permanent magnet 410 is moved closer to the side of the tube 100. In this case, the tube 100 and the permanent magnet 410 are not necessarily contact to each other, provided that the magnetic force generated by the permanent magnet 410 acts on the magnetic particles M within the tube 100. After the permanent magnet 410 is moved closer to the side of the tube 100, as shown in FIG. 15, the permanent magnet 410 is moved down in the longitudinal direction of the tube 100 while being rotated around the side of the tube 100 (A to H in the figure). As a result, the magnetic particles M pass through many regions of the reagents (the first washing solution forming the second plug 20 or the elution solution forming the fourth plug 40) provided as the plug surrounded by the inner wall of the tube 100. In this way, the magnetic particles M are washed with the reagent in an efficient manner.

In this embodiment, in order to move the magnetic particles M in the tube 100, what is required is to change the relative position between the permanent magnet 410 and the side of the tube 100 close to it. As described in the embodiments, the magnetic force application unit 400 having the permanent magnet(s) 410 may be moved around the side of the tube 100, or alternatively, the tube 100 may rotate on the longitudinal axis of the tube 100 rather than rotating the magnetic force application unit 400 around the tube 100. Alternatively, the rotation of the magnetic force application unit 400 may be combined with the rotation of the tube 100.

### Embodiment 3

A method of externally applying a magnetic force that is different from that described in the aforementioned embodiments is described according to the method of manipulation of the present invention.

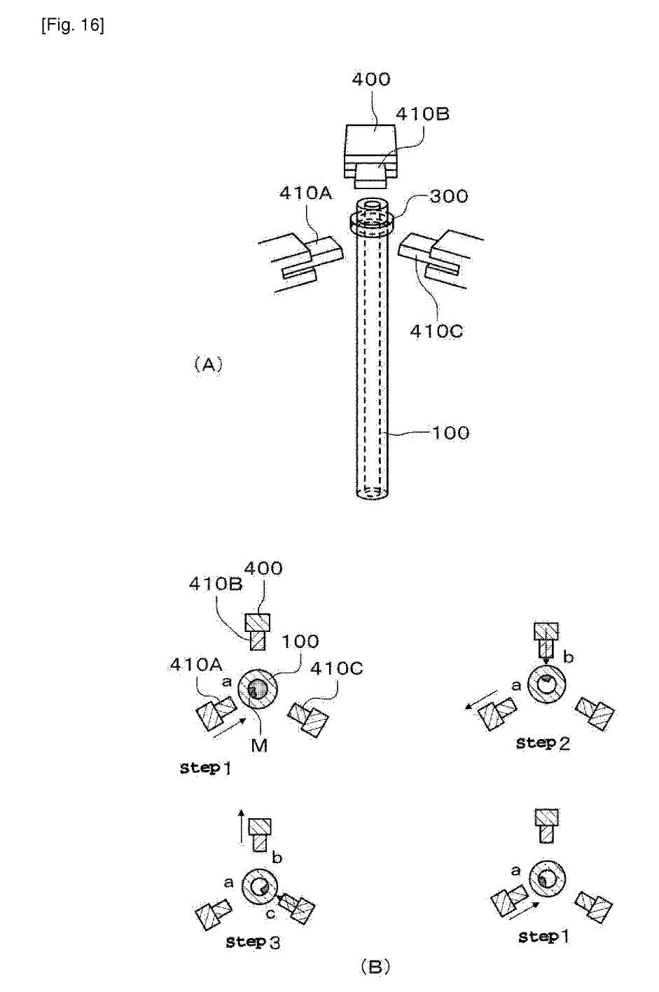

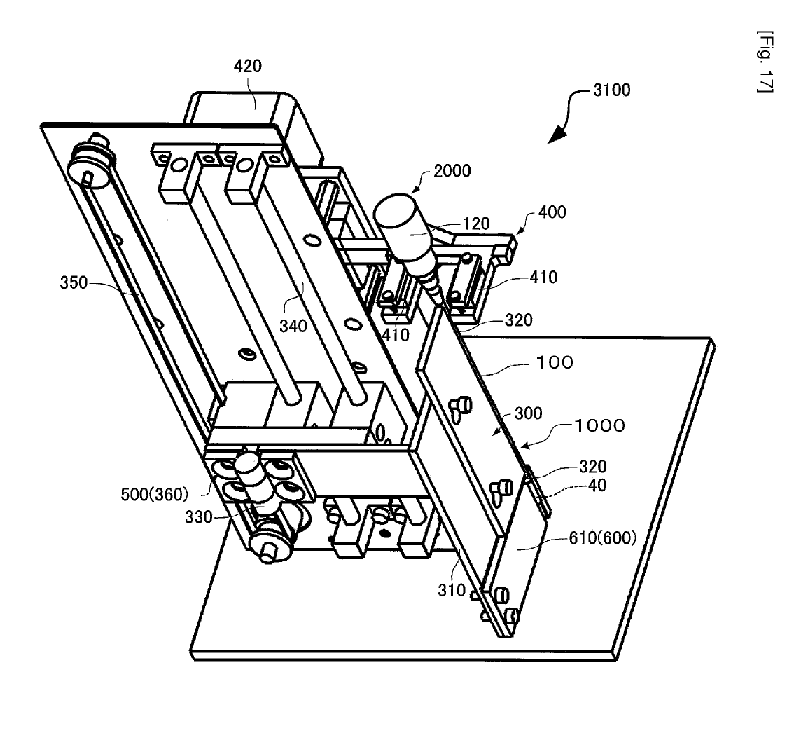

FIG. 16 is a view showing an exemplified configuration of the permanent magnets 410A, 410B, 410C and the tube 100 according to this embodiment. FIG. 16(A) shows a structure and FIG. 16(B) shows a moving procedure.

The permanent magnets 410A, 410B, 410C are connected to the magnetic force application unit 400. Either the permanent magnets 410A, 410B, 410C or the tube 100, or alternatively, both of them is/are connected to a moving mechanism that can change the relative position between them. In this embodiment, at least three permanent magnets 410A, 410B, 410C are used to move the magnetic particles M introduced into the tube 100 to at least three points in a cross-sectional plane perpendicular to the longitudinal direction of the tube 100, using the following procedures. In this way, as shown in FIG. 16(B), the magnetic particles M can be washed with the reagent more efficiently by allowing the magnetic particles M to pass through the regions of the reagent which otherwise they do not pass through when they merely travel in the liquid in the tube 100 between the permanent magnets.

Following is a description of a process of a method of externally applying a magnetic force for use in manipulating the magnetic particles M in the liquid in the tube 100 using at least three permanent magnets 410A, 410B, 410C. In this example, a distance between the tube 100 and any one of the three or more permanent magnets 410A, 410B, 410C is smaller than the distances between the tube 100 and the remaining permanent magnets.

1. At the step 1, the permanent magnet 410A is moved closer to near a first position, i.e., a point “a” on the side of the tube 100 and a relative distance with respect to the tube 100 is thus decreased. The remaining permanent magnets 410B, 410C are further away from the tube 100 than the permanent magnet 410A. The magnetic field is thus strongest at and near the point “a” around the tube 100.

2. At step 2, the permanent magnet 410A is moved away from the position near the point “a” and the relative distance with respect to the tube 100 is thus increased. On the other hand, the permanent magnet 410B is moved closer to near a second position, i.e., a point “b” on the side of the tube 100. The point “b” is different from the point “a” and is located at a position from which the magnetic field generated can reach the point “a” sufficiently. The relative distance between the permanent magnet 410B and the tube 100 is thus decreased. As in the step 1, the remaining permanent magnets 410A, 410C are away from the tube 100 at a longer distance than the permanent magnet 410B so that the magnetic field is strongest at and near the point “b” in the space around the tube 100.

3. At step 3, the permanent magnet 410B is moved away from the position near the point “b” and the relative distance with respect to the tube 100 is thus increased. On the other hand, the permanent magnet 410C is moved closer to near a third position, i.e., a point “c” on the side of the tube 100. The point “c” is different from the points “a” and “b” and is located at a position from which the magnetic field generated can reach the point “b” sufficiently. The relative distance between the permanent magnet 410C and the tube 100 is thus decreased. As in the step 1, the remaining permanent magnets 410A, 410B are away from the tube 100 at a longer distance than the permanent magnet 410C so that the magnetic field is strongest at and near the point “c” in the space around the tube 100.

4. The operation returns to the step 1. The point “a” on the side of the tube 100 to which the permanent magnet 410A approaches may be the same or different from the point “a” in the step 1. It should be noted, however, that the point “a” is a different position from the points “b” and “c” in the steps 2 and 3. In addition, it is preferable that the point “a” is located at a position from which the magnetic field generated can reach the point “c” sufficiently.

The magnetic particles M can be moved in the liquid in the tube 100 among at least three points in the plane perpendicular to the longitudinal direction of the tube 100 by moving the magnetic force application unit 400 having the permanent magnets 410A, 410B, 410C using the process described above. In addition, in the aforementioned steps, the magnetic force application unit 400 having the permanent magnets 410A, 410B, 410C is moved. However, either one or both of the magnetic force application unit 400 and the tube 100 may be moved as long as the relative position between each of the permanent magnet 410A, 410B, 410C and the tube 100 is altered in a similar manner. Furthermore, movement of the magnetic particles M in the longitudinal direction of the tube 100 may be made in addition to the movement of the magnetic particles M in the aforementioned cross-sectional plane of the tube 100. The magnetic particles M are successively moved through the reagents having different functions while passing through many regions in the liquid to do the intended washings.

While the embodiments have thus been described specifically, the present invention is not limited to the aforementioned embodiments and various other modifications can be made.

Modified Version 1

In the aforementioned embodiments, a combined motion is used in which the rotational movement of the permanent magnet 410 around the periphery of the tube 100 is combined with the relative movement to alter the distance between the permanent magnet 410 and the axis of the tube 100. As a result, the magnetic particles M travel along the inner surface of the side of the tube 100. By changing the distance between the permanent magnet 410 and the tube 100, the magnitude of the magnetic force around the tube 100 is also varied. The magnetic particles M in the tube 100 are repeatedly gathered and dispersed. As a result, the magnetic particles M reside in many regions in the reagents, which increases the efficiency of the washing.

Modified Version 2

Although the aforementioned embodiments describe and illustrate how the magnetic field is changed around the single tube 100, two or more tubes 100 may be arranged in parallel or in circle. A similar method of changing the magnetic force may be used to simultaneously manipulate the magnetic particles M in these tubes 100.

In this case, permanent magnets similar to the pair of permanent magnets 410A, 410B in the Embodiment 1 are arranged relative to the tubes 100 that are disposed in parallel. These two or more pairs of permanent magnets 410A, 410B are operated in a similar manner to those described above to simultaneously move the magnetic particles M in the tubes 100 in the back-and-force direction as well as in the up-and-down direction.

In addition, a permanent magnet is placed near the central axis of the circle formed by the tubes 100 that are arranged annually. A cylindrical permanent magnet is provided at a position outside the circle formed by the tubes 100. The outer permanent magnet moves in an eccentric path and the permanent magnet near the center moves in an eccentric path in synchronism with the outer permanent magnet. In this way, the magnitude of the magnetic field is changed over a period of time between the outer and inner sides of the tubes 100 that are arranged in circle.

Modified Version 3

Apparatus for the Extraction of Nucleic Acids

FIG. 17 is a perspective view showing an example of an apparatus for the extraction of nucleic acids according to this modified version. An apparatus for the extraction of nucleic acids 3100 is similar to the aforementioned apparatus for the extraction of nucleic acids 3000 except for a heating unit 600. The components and parts providing similar functions and operations are thus represented by like reference numerals, and description thereof will be omitted.

The heating unit 600 is for heating a part of the tube 100 after the tube 100 is mounted on the tube mount 300. The heating unit 600 may be, for example, a heat source and a heat block, a heater, and a coil for electromagnetic heating. The heating unit 600 may have any shape adapted to receive the tube 100 inserted thereinto or to contact with the side of the tube 100, as long as the heating unit 600 can heat the liquid in the tube 100.

A portion of the tube 100 heated by the heating unit 600 include a region where the fourth plug 40 is present in the longitudinal direction of the tube 100. The heating unit 600 may heat other portions of the tube 100. It is preferable, however, that the heating unit 600 does not heat the portion corresponding to the second plug 20 in the longitudinal direction of the tube 100.

In the apparatus for the extraction of nucleic acids 3100 shown in FIG. 17, a heater 610 is provided as the heating unit 600. The heater 610 is disposed in parallel to the support plate 310 and is adapted to heat a position including the fourth plug 40 in the tube 100. The heater 610 is in contact to an approximately half of the outer peripheral surface of the tube 100.

The apparatus for the extraction of nucleic acids 3100 can elute a sufficient amount of the nucleic acids into the elution solution forming the fourth plug 40 by means of the washing with at least one of the first washing solution forming the second plug 20 and the second washing solution forming the sixth plug 60 even when the amount of the nucleic acids adsorbed on the magnetic particles M is not so large. This contributes to increasing the washing efficiency and eluting the nucleic acids into the elution solution at a concentration sufficient for PCR.

Modified Version 4

Liquid Reservoir

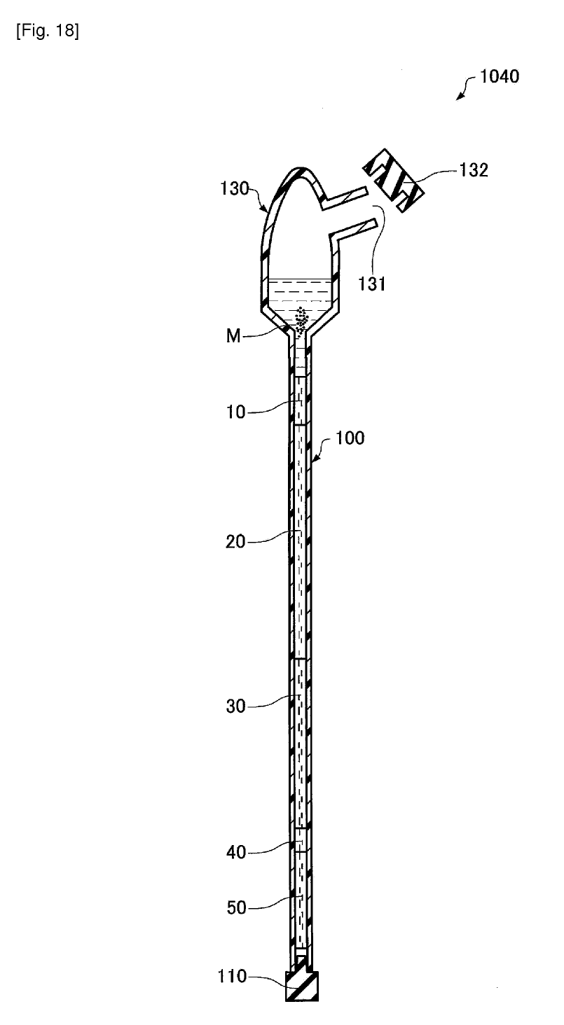

FIG. 18 is a view diagrammatically showing a device for the extraction of nucleic acids according to this modified version. As shown in FIG. 18, a device for the extraction of nucleic acids 1040 has a liquid reservoir (vessel) 130 formed at the end of the tube 100 at the side of the first plug 10 in communication with the tube 100. The liquid reservoir 130 is communicated with the tube 100.

The liquid reservoir 130 can hold liquid therein. The liquid reservoir 130 has an opening 131 through which a substance can be introduced into the liquid reservoir 130 from outside. The position at which the opening 131 is formed in the liquid reservoir 130 is not specifically limited. The liquid reservoir 130 may have two or more openings 131. The inner volume of the liquid reservoir 130 is not specifically limited, and may be, for example, equal to or larger than 0.1 mL but not larger than 100 mL. The material for use in making the liquid reservoir 130 is not specifically limited, and may be, for example, a polymer or a metal. It may be the same as the material used to make the tube 100.

By providing the liquid reservoir 130 as in the case of the device for the extraction of nucleic acids 1040, the magnetic particles M, the adsorption solution, and the sample can be contained in the liquid reservoir 130 to allow the magnetic particles M to adsorb the nucleic acids. These magnetic particles M can easily be introduced into the tube 100 through the end of the tube 100 at the side of the first plug 10.

In addition, the liquid reservoir 130 can be shaken along with the tube 100 to mix the liquid in the liquid reservoir 130 vigorously. This results in the rapid adsorption of the nucleic acids onto the magnetic particles M. Furthermore, by appropriately changing the amount of the sample to be introduced into the liquid reservoir 130 and the volume of the liquid in the tube 100, the nucleic acids in the sample can be concentrated quantitatively in the elution solution.

When the liquid reservoir 130 is provided as in the case of the device for the extraction of nucleic acids 1040, a lid 132 may be provided to seal the opening 131 in the liquid reservoir 130. The lid 132 is freely attached to the liquid reservoir 130 and removed therefrom. When a flexible and elastic material such as a rubber, an elastomer, or a polymer is selected as a material of the liquid reservoir 130, the inside of the tube 100 can be pressurized by deforming the liquid reservoir 130 with the lid 132 being fitted to the liquid reservoir 130.

This facilitates the application of a pressure to the tube 100 at the end thereof at the side of the first plug 10 in order to discharge the elution solution forming the fourth plug 40 having the nucleic acids eluted thereinto, from the tube 100 through the end at the side of the fifth plug 50. As a result, it becomes possible to perform from the step of introducing the sample into the liquid reservoir 130 to the step of easily removing the elution solution into, for example, a reaction chamber for PCR. In addition, the lid 132 can serve to prevent the liquid in the liquid reservoir 130 from being leaked therefrom when the liquid reservoir 130 is shaken along with the tube 100, which improves the efficiency of adsorbing the nucleic acids onto the magnetic particles M.

Modified Version 5

Variation of the Step of Moving the Magnetic Particles

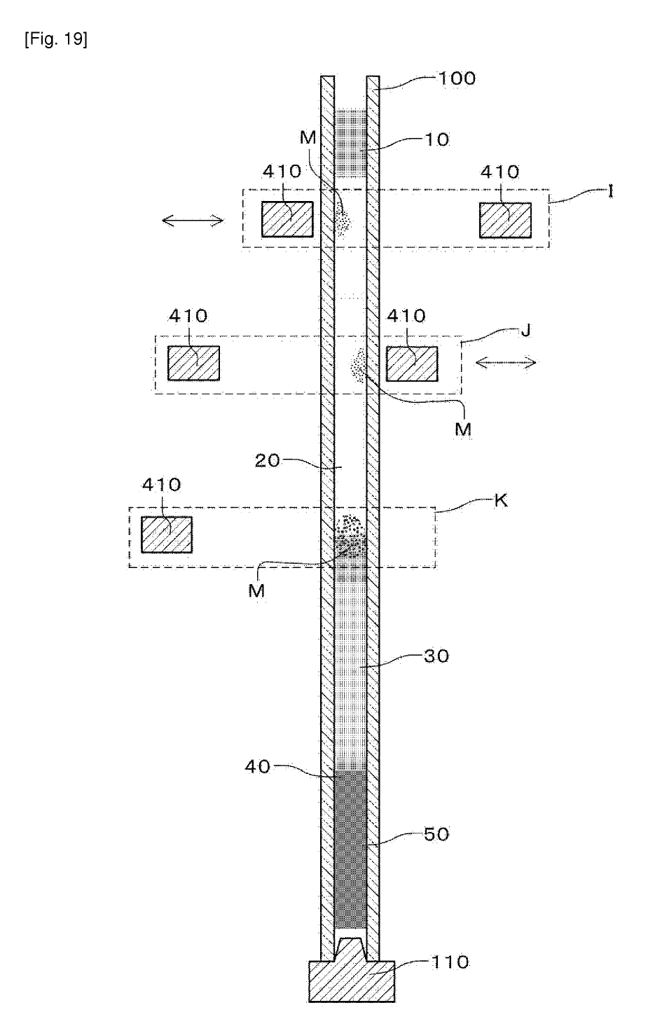

FIG. 19 is a diagrammatic representation for use in illustrating a method for the extraction of nucleic acids of this modified version.

In the aforementioned “4.4. Step of moving the magnetic particles,” the magnetic particles M are moved from the first plug to the fourth plug 40 through the intermediate plugs(s) by externally applying a magnetic force to the magnetic particles M. The magnetic force that is externally applied may, however, be varied to shake, collect, and disperse the magnetic particles M in the second plug 20 when the magnetic particles M are moved to the second plug 20. This can improve the effect of washing the magnetic particles M using the first washing solution forming the second plug 20.

More specifically, as shown in I and J in FIG. 19, when a pair of permanent magnets 410 is used as means for applying the magnetic force, the magnetic particles M are moved from the adsorption chamber 120 and passed through the first plug 10 using the permanent magnets 410. The magnetic particles M then reach the second plug 20. At that time, one of the permanent magnets 410 is moved away from the tube 100 and the other of the permanent magnets 410 is moved closer to the tube 100 from the opposite side. This results in the shaking of the magnetic particles M in the second plug 20 in the direction crossing the longitudinal direction of the tube 100 (repeated between I and J in the figure). This can improves the effect of washing the magnetic particles M using the first washing solution forming the second plug 20. This washing operation using the magnetic particles M may be applied to the second plug 20 and/or the sixth plug 60 in the case where the second plug 20 is divided into smaller plugs or in the case where the sixth plug 60 is provided within the tube 100.

In addition, as shown in K in FIG. 19, the magnetic particles M can be distributed in the second plug 20 merely by means of moving the permanent magnet 410 away from the tube 100. The magnetic particles M is less likely to be entered into the first plug 10 or the oil forming the third plug 30 even when they are distributed in the second plug 20 with a smaller magnetic force because each of the magnetic particles M has a hydrophilic surface. Therefore, such an aspect may also be used.

More specifically, when the magnetic particles M reach the second plug 20 after the magnetic particles M are moved from the adsorption chamber 120 and passed through the first plug 10 using the permanent magnets 410, the permanent magnets 410 are moved away from the tube 100 to distribute the magnetic particles M in the second plug 20. Then, the magnetic particles M are again moved and passed through the third plug into the fourth plug 40 using the magnetic force generated by the permanent magnets 410.

The aspect in which the magnetic force that is externally applied is changed to shake and repeatedly collect and disperse the magnetic particles M may be applied to a case where the magnetic particles M are present in the adsorption solution in the adsorption chamber 120 or a case where the magnetic particles M are present in the fourth plug 40 (the elution solution).

Modified Version 6

Variation of the Step of Eluting the Nucleic Acids

The aforementioned “4.5. Step of eluting the nucleic acids” may be performed while heating the fourth plug 40. The fourth plug 40 can be heated using one of appropriate methods including, for example, by contacting a thermal medium such as a heat block to a position corresponding to the fourth plug 40 in the tube 100, by using a heat source such as a heater, or using electromagnetic heating.

When the fourth plug 40 is heated, the plugs other than the fourth plug 40 may also be heated. It is, however, preferable that the plug of the washing solution is not heated if the magnetic particles M carrying the nucleic acids adsorbed thereon are present in the plug of the washing solution. The temperature to be reached while heating the fourth plug 40 is preferably equal to or higher than 35° C. but not higher than 85° C., more preferably, equal to or higher than 40° C. but not higher than 80° C., and yet more preferably, equal to or higher than 45° C. but not higher than 75° C., in terms of the efficiency of the elution as well as in terms of inhibiting deactivation of an enzyme for PCR when the enzyme is contained in the elution solution.

In the step of eluting the nucleic acids, it is possible to elute the nucleic acids carried by the magnetic particles M into the elution solution in a more efficient manner with the fourth plug 40 being heated. In addition, it is possible to elute, into the elution solution, the nucleic acids that are left on the magnetic particles M without having been eluted in the washing solution even when the composition of the first washing solution or the second washing solution is identical or similar to the composition of the elution solution. In other words, the nucleic acids can further be eluted into the elution solution after the magnetic particles M having the nucleic acids adsorbed thereon are washed with the first washing solution or the second washing solution. It is thus possible to achieve both the sufficient washing and the elution at a sufficient concentration into the elution solution even when the composition of the washing solution is identical or similar to the composition of the elution solution.

Modified Version 7

Variation of the Step of Discharging the Fourth Plug from the Tube

When the aforementioned “4.6. Step of discharging the fourth plug from the tube” is used, the magnetic particles M after the elution of the adsorbed nucleic acids into the elution solution in that step may be present in the fourth plug 40. Alternatively, the magnetic particles M may be transferred to either one of the first plug 10, the second plug 20, and the third plug 30 or to the adsorption chamber 120 by further applying a magnetic force. This process makes it possible to discharge the fourth plug 40 from the tube 100 without containing any magnetic particles M in the elution solution. When the magnetic particles M is transferred to the second plug 20 or the adsorption chamber 120, the magnetic particles M are less likely to enter the oil forming the third plug 30 after the magnetic force is removed. Accordingly, the fourth plug 40 can be discharged more easily from the tube 100.

The present invention is not limited to the aforementioned, embodiments, and various other modifications can be made. For example, the present invention includes a configuration that is substantially similar to the configurations described in the embodiments (such as a configuration that is identical in function, method, and result or a configuration that is identical in object and effect). In addition, the present invention includes a configuration in which a non-essential part of the configuration described in one embodiment has been replaced with. Furthermore, the present invention includes a configuration that has the same effect or that achieves the same object as the configuration described in one embodiment. Moreover, the present invention includes a configuration that is a combination of the configuration described in the embodiments with a known technique.

1. A method of manipulating a solid carrier in a liquid contained in a vessel, the solid carrier being magnetic and capable of carrying a substance, the method comprising:
manipulating the solid carrier using a magnetic force application unit adapted to applying a magnetic force to the solid carrier in the vessel so that the relative position between the magnetic force application unit and the vessel is altered over a period of time by means of directing the solid carrier in a direction along either one or a combination of the vectors of the longitudinal direction of the vessel and a cross-sectional direction crossing the longitudinal direction of the vessel.

2. The method of manipulating a solid carrier according to claim 1, wherein:
the magnetic force application unit comprises at least one permanent magnet.

3. The method of manipulating a solid carrier according to claim 2, wherein:
the position of the solid carrier is changed in the vessel by moving the permanent magnet to a position of the peripheral surface of the vessel and then moving the permanent magnet away from the position to move the permanent magnet closer to another position of the peripheral surface of the vessel.

4. The method of manipulating a solid carrier according to claim 2, wherein:
the solid carrier is directed using two permanent magnets that are opposed to each other at a certain distance.

5. The method of manipulating a solid carrier according to claim 1, wherein:
the solid carrier is directed within the vessel as a result of the relative rotation between the magnetic force application unit and the vessel.

6. The method of manipulating a solid carrier according to claim 2, wherein:
at least three permanent magnets are provided, and the solid carrier is directed in at least two directions in a plane perpendicular to the longitudinal direction of the vessel as a result of the relative displacement of the position between the vessel and the permanent magnets.

7. An apparatus of manipulating a solid carrier in a liquid contained in a vessel, the solid carrier being magnetic and capable of carrying a substance, the apparatus comprising:
a magnetic force application unit adapted to applying a magnetic force to the solid carrier in the vessel, the solid carrier being manipulated so that the relative position between the magnetic force application unit and the vessel is altered over a period of time by means of directing the solid carrier in a direction along either one or a combination of the vectors of the longitudinal direction of the vessel and a cross-sectional direction crossing the longitudinal direction of the vessel.

8. A method of manipulating a magnetic solid carrier in a vessel comprising liquid, the vessel having a longitudinal direction, the method comprising the step of:
altering a relative position between the magnetic force application unit adapted to applying a magnetic force to the solid carrier from outside the vessel and the vessel is altered over a period of time, to direct the solid carrier, in one of a planar direction crossing the longitudinal direction of the vessel, the longitudinal direction of the vessel, and a direction determined by combining the vectors of the longitudinal direction of the vessel and the planar direction crossing the longitudinal direction of the vessel.

9. The method of manipulating a solid carrier according to claim 8, wherein:
the magnetic force application unit comprises at least one permanent magnet.

10. The method of manipulating a solid carrier according to claim 8, comprising:
moving the magnet closer to a first position of the vessel;
moving the magnet away from the first position;
moving the magnet closer to a second position of the vessel, the second position being different from the first position; and
moving the magnet away from the second position; wherein the solid carrier is directed in the vessel thereby.

11. The method of manipulating a solid carrier according to claim 9, wherein:
the magnet is two permanent magnets that are opposed to each other at a certain distance.

12. The method of manipulating a solid carrier according to claim 11, comprising
decreasing a relative distance between a first magnet and the vessel while increasing a relative distance between a second magnet and the vessel, the second magnet being opposed to the first magnet; and
increasing the relative distance between the first magnet and the vessel while decreasing the relative distance between the second magnet and the vessel;
wherein the solid carrier is directed in the vessel thereby.

13. The method of manipulating a solid carrier according to claim 8, wherein:
the solid carrier is directed in the vessel by means of rotating the magnetic force application unit around the vessel.

14. The method of manipulating a solid carrier according to claim 9, wherein:
at least three permanent magnets are provided, and one of a first distance, second distance, and a third distance being determined to be smaller than the remaining two distances, the first distance representing a distance between the vessel and a first magnet, the second distance representing a distance between the vessel and a second magnet, and the third distance representing a distance between the vessel and a third magnet; and the solid carrier is directed in a plane perpendicular to the longitudinal direction of the vessel thereby.

15. An apparatus of manipulating a magnetic solid carrier in a vessel comprising liquid, the vessel having a longitudinal direction, the apparatus comprising:
magnetic force application unit adapted to applying a magnetic force to the solid carrier from outside the vessel, wherein the magnetic force application unit is manipulated so that the relative position between the magnetic force application unit and the vessel is altered over a period of time, to direct the solid carrier, by using the magnetic force application unit, in one of a planar direction crossing the longitudinal direction of the vessel, the longitudinal direction of the vessel, and a direction determined by combining the vectors of the longitudinal direction of the vessel and the planar direction crossing the longitudinal direction of the vessel.

16. The apparatus of manipulating a solid carrier according to claim 15, wherein:
the vessel is a tube having a first oil plug, a washing solution plug, a second oil plug, an elution solution plug, and a third oil plug in the longitudinal direction thereof.
